(19) United States
(12) Patent Application Publication
(10) Pub. No.: US 2026/0069578 A1
(43) Pub. Date: Mar. 12, 2026

(54) TREATMENT OF NEUTROPHILIC DERMATOSES

(71) Applicant: UNION THERAPEUTICS A/S, Hellerup (DK)

(72) Inventors: Elisabeth Hjardem TAUDORF, Roskilde (DK); Gregor B.E. JEMEC, Roskilde (DK); Kim KJØLLER, Alsgarde (DK)

(21) Appl. No.: 18/857,955

(22) PCT Filed: Apr. 18, 2023
(86) PCT No.: PCT/EP2023/060001

§ 371 Date: Oct. 18, 2024

(30) Foreign Application Priority Data
Apr. 19, 2022 (GB) 2205715.2

(51) Int. Cl. A61K 31/4436; A61K 9/20; A61K 9/48; A61K 45/06; A61P 17/02; A61P 17/10

(52) U.S. Cl. CPC A61K 31/4436; A61K 9/2054; A61K 9/4866; A61K 45/06; A61P 17/02; A61P 17/10

(57) ABSTRACT

Provided is a PDE4 inhibitor, in particular orismilast or a pharmaceutically acceptable salt, solvate or hydrate thereof, for use in the treatment of neutrophilic dermatoses in a subject. In particular, the invention provides orismilast or a pharmaceutically acceptable salt, solvate or hydrate thereof, for use in the treatment of pyoderma gangrenosum.

This invention relates to the use of a PDE4 inhibitor, in particular orismilast, in the treatment of one or more clinical signs or symptoms of neutrophilic dermatoses, such as pyoderma gangrenosum, in a subject.

### BACKGROUND

Pyoderma gangrenosum is a rare, non-infectious inflammatory skin disease characterized by reddish or purple papules or nodules that develop into swollen, open sores or ulcerations having a well-defined blue or violet-coloured border. The ulcerations can spread, widen and deepen and can be very painful. The disorder can greatly impact quality of life, can cause extensive scarring, and may cause significant morbidity in affected patients. The condition can affect any area of the body. There are several forms of PG that differ by their clinical presentation, location and associated diseases, including classic PG, atypical/bullous PG, pathergic PG, necrotizing-fasciitis-like PG, pustular PG, vegetative PG, malignant PG, peristomal PG and post-operative PG.

Classic pyoderma gangrenosum can occur on any skin surface but most often occurs on the legs and is characterized by deep ulcerations. These lesions often begin as small pus-filled bumps (pustules) that enlarge and spread rapidly. This form of the disease is often very painful and patients may also feel unwell with symptoms such as fever, malaise, arthralgia and myalgia. Classic pyoderma gangrenosum also occurs near surgical openings (e.g. stoma sites) in the body. This condition is referred to as peristomal pyoderma gangrenosum.

Atypical or bullous pyoderma gangrenosum is characterized by superficial blisters (bullae) that spread rapidly in a concentric pattern. This form of the disease most often affects the hands and is often associated with an underlying haematological disorder such as leukaemia.

Pustular pyoderma gangrenosum is a rare variant of the disease characterized by painful bumps (pustules) most often found on the trunk, arms and legs. These lesions eventually develop into ulcerations. This form of the disease is often associated with inflammatory bowel disease.

Vegetative pyoderma gangrenosum is a superficial form of the disease which is characterized by chronic ulcerations that are not usually painful. It often presents as a single lesion in patients who are otherwise healthy.

Malignant PG is a rare variant that typically presents with severe ulceration of the torso, head and neck. This form of PG is not associated with systemic disease.

Post-operative (or post-surgical) pyoderma gangrenosum has a lower association with systemic disease than other forms of pyoderma gangrenosum. Post-operative pyoderma gangrenosum has been reported to occur most frequently after breast, cardiothoracic, abdominal and obstetric surgery. It can often be misdiagnosed as infection of the surgical site, causing delay to wound healing.

Dysregulation of both the innate and the adaptive immune system is thought to play a role in PG. Innate immune signalling pathways including the pattern recognition receptor (PRR), Janus kinase (JAK) 1-3 and signal transducer and activator of transcription (STAT) pathways have been found to be upregulated in lesional skin compared with non-lesional skin in patients with PG (Ortega-Loayza et al., Br. J. Dermatol. 2018; 178(1):e35-e36; Alves de Mederros et al., PLoS One. 2016; 11(10):e0164080). Both individuals with classic PG and those with PASH syndrome (PG, acne, and suppurative hidradenitis) have shown overexpression of IL-1β, tumor necrosis factor alpha (TNFα), IL-17, endothelial- and leukocyte-selectin, and chemokines IL-8, OXCL16, and RANTES (Marzano et al., Clin. Exp. Immunol., 2014; 178(1):48-56; Marzano et al., Br. J. Dermatol. 2017:176(6):1588-1598). as well as matrix metalloproteinases (MMPs) (Braswell et al., J. Am. Acad. Dermatol., 2015; 73(4):691-8). Biopsies of pre-lesional PG papules have shown CD3+ infiltrates and increased inflammatory cytokines, while PG lesions have shown significant overexpression of IL-1α, IL-1β, IL-6, IL-8, and IL-36α (Kolios et al., Br. J. Dermatol. 2015: 173(5):1216-23: Wang et al., Front. Immunol. 2018;81980). The adaptive immune system is also thought to play a role in PG, since therapeutics which interfere with T-cell function and promoting apoptosis have been found to improve symptoms.

Neutrophilic dermatoses are a heterogenous group of non-infectious anti-inflammatory diseases, including pyoderma gangrenosum (PG), which are characterised by infiltration of the epidermis, dermis and/or hypodermis by neutrophils. Interleukin-6 (IL-6), a pro-inflammatory cytokine that plays a role in activation and accumulation of neutrophils, has been found to be elevated in PG lesions.

Neutrophilic dermatoses tend to be associated with distinct underlying systemic diseases, in particular inflammatory bowel disease and haematological malignancies. For example, about half of PG cases are associated with underlying systemic conditions such as inflammatory bowel disease (e.g. Crohn's disease or ulcerative colitis), inflammatory arthritis and haematological malignancies (for example leukaemia and IgA monoclonal gammopathy).

There is no single specific treatment for PG and other forms of neutrophilic dermatoses. Treatment may include analgesia, wound care and/or compression therapy alongside topical and/or systemic therapy, which typically includes administration of steroids and/or immunosuppressants, for example cyclosporin or tacrolimus. Biologics, such as TNFα inhibitors, antagonists of IL-1, IL-12, IL-23 and IL-6, JAK/STAT inhibitors and immunoglobulin therapy, have also been proposed for the treatment of PG.

Apremilast, an oral PDE4 inhibitor, has reportedly been used in one case of resistant vegetative PG with underlying IgA kappa monoclonal gammopathy of unknown significance (MGUS). Complete healing of one lesion and partial healing of another was observed when apremilast was used concomitantly with oral prednisone (Laird et al., JAAD Case Rep. 2017; 3(3):228-229),

Phosphodiesterases (PDEs) are the only enzymes that hydrolyze and degrade cAMP. PDE4 is a cAMP phosphodiesterase widely expressed in hematopoietic cells (e.g. myeloid, lymphoid), nonhematopoietic cells (e.g. smooth muscle, keratinocyte, endothelial), and sensory/memory neurons. The four PDE4 genes (A, B, C, and D) exhibit distinct target and regulatory properties. Each of these genes can produce multiple protein products due to mRNA splice variants, resulting in approximately 19 different PDE4 proteins that fall into either short or long isoform categories. Long isoforms are differentiated from short isoforms by an additional upstream conserved region (UCR), which contains a PKA activation site. These UCR sequences play a critical role in the regulation of PDE4 through the phosphorylation of PKA and extracellular signal-regulated kinase (ERK). For example, the major PDE4 isoforms expressed in leukocytes are PDE4 B2 (short isoform) and PDE4 D3 and D5 (long isoforms). Long PDE4 D isoforms predominate in monocytes, whereas short PDE4 B isoforms predominate in macrophages. The catalytic activity of PDE4 B2 is activated by ERK phosphorylation, whereas the catalytic function of the D3 and D5 variants is inhibited by ERK activation. Thus, the UCR modules can determine the functional outcome of ERK phosphorylation. This means that the pro-inflammatory mediators of monocytes trigger an overall decrease in PDE4 activity, whereas the proinflammatory mediators of macrophages trigger an overall increase in PDE4 activity.

As cAMP is a key second messenger in the modulation of inflammatory responses, PDE4 has been found to regulate inflammatory responses of inflammatory cells by modulating proinflammatory cytokines such as TNF-a, IL-2, IFN-γ, GM-CSF and LTB4. Inhibition of PDE4 has therefore become an attractive target for the therapy of inflammatory diseases, although it is associated with significant side effects, particularly nausea and emesis (Lagente V et al., Mem Inst Oswaldo Cruz, Rio de Janeiro, 2005 v.100(Suppl. I): 131-136; Shett G et al., Ther Adv Musculoskel Dis, 2010, v 2(5) 271-278).

Neutrophilic dermatoses, such as PG, are difficult to treat and no cure exists for these conditions. Medical interventions that are both therapeutically efficacious and safe are therefore highly desirable. There remains a need for effective treatments for PG and other forms of neutrophilic dermatoses.

### BRIEF SUMMARY OF THE DISCLOSURE

According to a first aspect of the invention, there is provided a compound of formula (I):

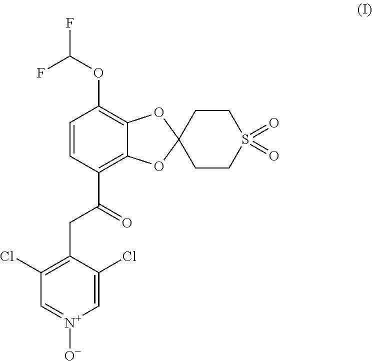 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof for use in the treatment of neutrophilic dermatoses, such as PG, in a subject. The compound of formula (I) is 2-(3,5-Dichloro-1-oxido-pyridine-4-yl)-1-(7-difluoromethoxy-2′,3′,5′,6′-tetrahydro-spiro[1,3-benzodioxole-2, 4′-(4H)-thiopyran-1′,1′-dioxide]-4-yl)ethenone, which is also known as orismilast.

In some embodiments, the treatment comprises the use of a pharmaceutically acceptable salt of the compound of formula (I).

According to a second aspect of the invention, there is provided a compound of formula (I) as defined herein, or a pharmaceutically acceptable salt, solvate or hydrate thereof, for use in reducing pain caused by or associated with neutrophilic dermatoses, such as PG.

According to a third aspect of the invention, there is provided a compound of formula (I) as defined herein, or a pharmaceutically acceptable salt, solvate or hydrate thereof, for use in reducing inflammation caused by or associated with neutrophilic dermatoses, such as PG.

The neutrophilic dermatoses may be selected from pyoderma gangrenosum (PG, including pustular PG, atypical/bullous PG, vegetative PG, pathergic PG, necrotizing-fasciitis-like PG, peristomal PG, and post-operative PG), Sweet's syndrome (SS, also known as acute febrile neutrophilic dermatosis, including bullous SS, pustular SS, giant cellulitis-like SS, necrotizing fasciitis-like SS, drug-induced SS and subcutaneous SS), Sneddon-Wilkinson disease (also known as subcorneal pustular dermatosis), Behget disease, neutrophilic panniculitis, neutrophilic eccrine hidradenitis, erythema elevatum diutinum, neutrophilic urticaria, Group of IgA neutrophilic dermatosis, amicrobial pustulosis of the folds, Hallopeau's continuous acrodermatitis, acute generalised exanthematous pustulosis, infantile acropustulosis, aseptic abscesses, PASH syndrome (pyoderma gangrenosum, acne and HS), PAPA syndrome (pyoderma gangrenosum, acne and pyogenic arthritis), PASS syndrome (PG, acne conglobate, HS, seropositive spondyloarthropathies), PAPASH syndrome (PG, pyogenic arthritis, acne, HS), PsAPASH (psoriatic arthritis, PG, acne, HS) histiocytoid neutrophilic dermatitis, neutrophilic dermatitis of the dorsal hands, bowel bypass syndrome (bowel-associated dermatitis-arthritis syndrome), palisading neutrophilic granulomatous dermatitis, VEXAS syndrome, or any combination thereof.

In some embodiments the neutrophilic dermatoses is pyoderma gangrenosum.

In some embodiments the pyoderma gangrenosum (PG) is selected from classic PG, atypical/bullous PG, pustular PG, vegetative PG, pathergic PG, necrotizing-fasciitis-like PG, malignant PG, peristomal PG and post-operative PG.

In some embodiments, the subject has concomitant hidradenitis suppurativa (HS).

In some embodiments the subject has PASH, PAPA, PASS, or PsAPASH syndrome.

In some embodiments the subject has PASH syndrome.

In some embodiments the subject is not suffering from hidradenitis suppurativa (HS) as the sole form of neutrophilic dermatoses. It will thus be understood that the subject may be suffering from another form of neutrophilic dermatoses (e.g. pyoderma gangrenosum) which is concomitant or associated with HS, but that the subject is not suffering from HS alone, i.e. in the absence of a further form of neutrophilic dermatoses.

In some embodiments the subject does not have HS.

The treatment may comprise oral, topical and/or intravenous administration of the compound of formula (I), or a pharmaceutically acceptable salt, solvate or hydrate thereof. In some embodiments the treatment comprises oral administration. In some embodiments the treatment comprises topical administration. In some embodiments the treatment comprises oral and topical administration.

The treatment may be administered for at least 10 days, at least 2 weeks, at least 3 weeks, at least 4 weeks, at least 6 weeks or at least 8 weeks. In some embodiments, the treatment is administered for no more than 26 weeks, no more than 20 weeks, no more than 16 weeks, no more than 12 weeks or no more than 10 weeks. It will be appreciated that, in some cases, chronic treatment may be required. Thus, in some embodiments treatment is administered for at least 12 weeks, at least 16 weeks, at least 20 weeks, at least 6 months, at least 8 months, at least 10 months or at least 12 months. In some embodiments the treatment is administered for a period of time of from 6 months to 5 years, from 12 months to 4 years, from 15 months to 3 years, or from 18 to 24 months.

The treatment may comprise administering a total daily dose of no more than 120 mg, no more than 100 mg, no more than 80 mg, no more than 60 mg, or no more than 40 mg of the compound of formula (I). In some embodiments, the treatment comprises administering a total daily dose of at least 5 mg, at least 10 mg, at least 20 mg, at least 30 mg, at least 40 mg, at least 50 mg or at least 60 mg. For example, the total daily dose administered may be from about 5 to about 120 mg, from about 10 to about 120 mg, from about 20 to about 110 mg, from about 30 to about 100 mg, from about 40 to about 90 mg, from about 50 to about 80 mg, or from about 60 to about 70 mg. In some embodiments the total daily dose of the compound of formula (I) administered is from about 50 to about 90 mg, from about 60 to about 80 mg. In some embodiments the treatment comprises administering a total daily dose of about 40 mg of the compound of formula (I). In some embodiments the treatment comprises administering a total daily dose of about 5 mg of the compound of formula (I). In some embodiments the treatment comprises administering a total daily dose of about 10 mg of the compound of formula (I). In some embodiments the treatment comprises administering a total daily dose of about 20 mg of the compound of formula (I). In some embodiments the treatment comprises administering a total daily dose of about 60 mg of the compound of formula (I). In some embodiments the treatment comprises administering a total daily dose of about 80 mg of the compound of formula (I). In some embodiments the treatment comprises administering a total daily dose of about 100 mg of the compound of formula (I).

In some embodiments, the treatment comprises administering a dose of from about 5 to about 70 mg, from about 10 to about 60 mg, from about 15 to about 50 mg, or from about 20 to about 40 mg, e.g. about 20 mg, about 30 mg or about 40 mg.

The treatment may be administered from one to four times daily, for example from two to three times daily. In some embodiments the treatment is administered once daily. In some embodiments the treatment is administered twice daily.

The compound of formula (I) as defined herein, or a pharmaceutically acceptable salt, solvate or hydrate thereof, may be comprised within a composition or formulation. Thus, the invention also provides a composition or formulation comprising a compound of formula (I), or a pharmaceutically acceptable salt, solvate or hydrate thereof, for use in the treatment of neutrophilic dermatoses, for example PG. The composition or formulation may be formulated according to the desired route of administration.

In some embodiments, the compound, or a composition or formulation comprising the compound, is formulated for oral administration. Suitably, the compound, composition or formulation is formulated in the form or a tablet or capsule. In some embodiments, the compound is comprised within a modified-release formulation.

The subject may be a human or an animal. In some embodiments the subject is a human. In some embodiments the subject is a human male. In some embodiments the subject is a human female.

In some embodiments, the subject is suffering from a comorbidity selected from obesity, metabolic syndrome, inflammatory bowel disease (such as Crohn's disease or ulcerative colitis), spondyloarthropathy, inflammatory arthritis, a haematological malignancy (such as leukaemia (e.g. acute myeloid leukaemia) or IgA monoclonal gammopathy), hepatitis (e.g. hepatitis C), blood dyscrasia, granulomatosis with polyangiitis, psoriasis, atopic dermatitis, or any combination thereof.

The treatment may be administered in combination with a further therapy. The further therapy may be selected from an anti-androgenic agent, a hormone, an antibiotic, a retinoid, an anti-inflammatory agent (including steroids and non-steroidal anti-inflammatory agents), an analgesic, an immunosuppressive agent, an antibody, surgery or any combination thereof.

In some embodiments the treatment provides selective inhibition of PDE4D and/or PDE4B. In certain embodiments the treatment provides selective inhibition of PDE4B2, PDE4B3, PDE4D2, PDE4D3, PDE4D4, PDE4D5 and/or PDE4D7. In further embodiments, the treatment may provide selective inhibition of PDE4D3 and/or PDE4B2.

### DETAILED DESCRIPTION

The invention will now be described by way of example and with reference to the accompanying Figures, in which:

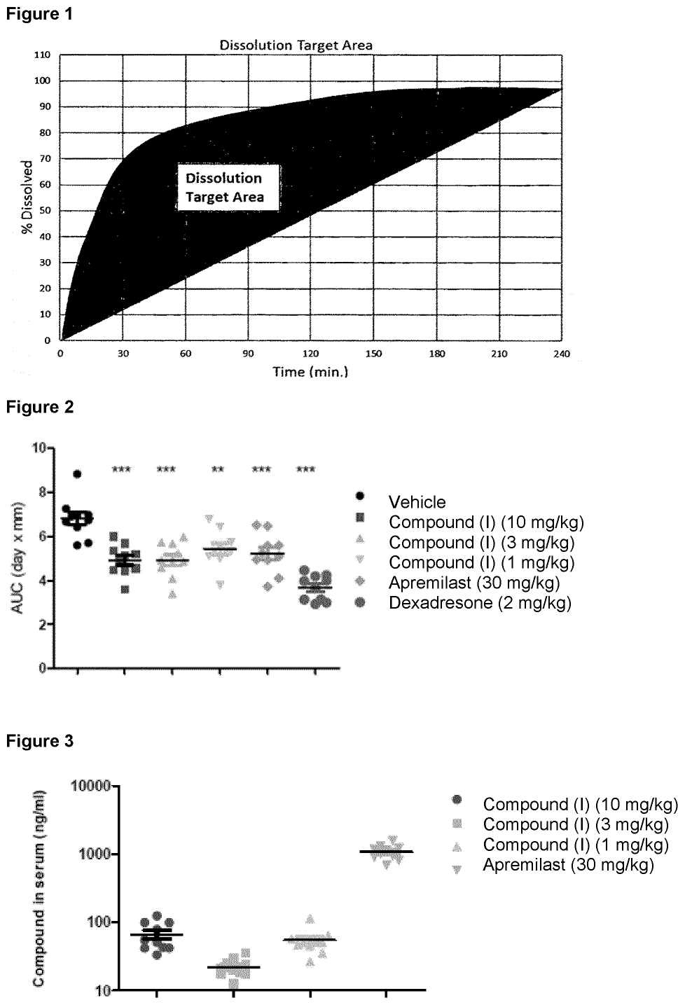

FIG. 1 is a chart illustrating the dissolution target area for a modified release formulation; dissolution method: Paddle 75 rpm, 900 ml 0.1N HCl+0.5% SDS, 37° C., HPLC detection;

FIG. 2 shows the AUC for ear thickness in mice dosed with the compound of formula (I) or apremilast; and

FIG. 3 shows the concentration of compound in serum in mice dosed with the compound of formula (I) or apremilast.

### DEFINITIONS

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the subject; slowing in the rate of degeneration or decline; making the final point of degeneration less debilitating; improving the physical or mental well-being of the subject. For example, in relation to neutrophilic dermatoses, such as pyoderma gangrenosum, “treatment” may comprise one or more of: eliminating, promoting healing of, or reducing the severity, spread, size, depth, growth rate and/or number of, inflammatory nodules, blisters, pustules, abscesses, comedones, ulcers and/or sinus tracts; promoting wound healing, or increasing the rate of healing; reducing or eliminating pain, inflammation, burning, swelling, redness, itching and/or discomfort; reducing or eliminating discharge; preventing or reducing scarring and/or disfigurement; preventing or reducing the likelihood and/or severity of secondary infection (e.g. bacterial infection), cancer (e.g. squamous cell carcinoma), septicaemia and/or anaemia; preventing or minimizing the incidence of depression and other psychological effects; controlling any underlying disease; preventing or delaying the progression of disease; a reduction in the disease severity according to the Dermatology quality of Life Index (DLQI), the European quality of life—5 Dimensions (EQ-5D) scale, the Dermatology Life Quality Index (DLQI), the McGill Pain questionnaire, the Visual analog scale of pain (VAS pain), the Work Productivity and Activity Impairment Questionnaire: Specific Health Problem V2.0 (WPAI:SHP), the Anxiety and depression (HADS) questionnaire and/or the Multidimensional Fatigue Inventory 20 (MFI-20). The compound of formula (I) has a rapid onset of action and may therefore provide rapid relief of one or more symptoms and/or clinical features of neutrophilic dermatoses, such as pyoderma gangrenosum. For example administration of the compound of formula (I) to a subject with neutrophilic dermatoses, such as pyoderma gangrenosum, may provide rapid relief of pain associated with the condition.

When a compound or salt, solvate or hydrate described in this specification is administered to treat a disorder, a “therapeutically effective amount” is an amount sufficient to reduce or completely alleviate symptoms or other detrimental effects of the disorder; cure the disorder; reverse, completely stop, or slow the progress of the disorder; or reduce the risk of the disorder getting worse.

The term “pharmaceutically acceptable salt” refers to salts that retain the biological effectiveness and properties of the compounds described herein, and which are not biologically or otherwise undesirable. Pharmaceutically acceptable salts of compound (I) are well known to skilled persons in the art. It may be that a reference to a salt of compound (I) herein may refer to a pharmaceutically acceptable salt of compound (I). The invention covers all crystalline modifications, polymorphic forms and mixtures thereof. In some embodiments, the treatment comprises administration of the polymorphic form E of the compound of formula (I).

The term “solvate” is intended to indicate a species formed by interaction between a compound, e.g. a compound of formula (I), and a solvent, e.g. alcohol, glycerol or water, wherein said species are in a solid form. When water is the solvent, said species is referred to as a “hydrate”.

The phrase “phosphodiesterase” as used herein refers to one or more of the phosphodiesterases (PDEs), PDE4, PDE7 and PDE8 being selective for cAMP. PDE4 is the most important modulator of cAMP. PDE4 is cAMP-specific and the dominant PDE in inflammatory cells. PDE4 enzymes are encoded by four genes (PDE4A, PDE4B, PDE4C and PDE4D), each of which is capable of producing a number of isoforms through mRNA splicing and the use of different promoters. Each PDE4 isoform within a particular PDE4 sub-family comprises a common core region, consisting of the catalytic unit and the C-terminal portion, and is defined by its unique N-terminal region. The PDE4 isoforms are further classified as long, short or super-short, depending on the presence or absence (or truncation) of two highly conserved sequences: Upstream Conserved Region 1 (UCR1) and Upstream Conserved Region 2 (UCR2). “Long” isoforms comprise both UCR1 and UCR2; “short” isoforms lack UCR1 and “super-short” isoforms lack UCR1 and have a truncated UCR2.

The phrase “PDE inhibitor” as used herein refers to a substance which inhibits PDE.

References to “topical treatment” or “topical administration” refer to the application of the compound, or a formulation comprising the compound, to the skin, soft tissues or mucous membranes.

Reference to a “subject” herein means a human or animal subject. Preferably the subject is warm-blooded mammal. More preferably the subject is a human.

Unless stated otherwise, reference herein to a “% by weight of the compound of formula (I), or a pharmaceutically acceptable salt, solvate or hydrate thereof” is intended to refer to the amount of the non-salt form of the compound. For example, reference to a composition comprising “5% by weight of the compound of formula (I) or a pharmaceutically acceptable salt, solvate or hydrate thereof” refers to a composition comprising 5% by weight of the compound in the non-salt form. Accordingly, where such a composition comprises a pharmaceutically acceptable salt the compound, the absolute amount of the salt in the composition will be higher than 5% by weight in view of the salt counter ion that will be also be present in the composition.

Reference to “about” in the context of a numerical is intended to encompass the value +/− 10%. For example, about 20% includes the range of from 18% to 22%.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Compound of Formula (I)

The present invention relates to a formulation comprising a compound of formula (I):

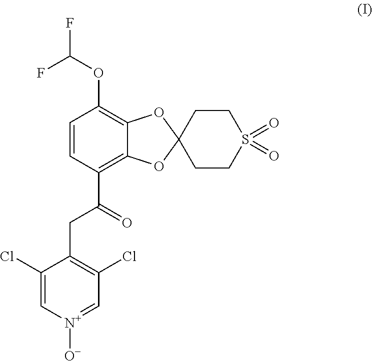 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof.

The compound of formula (I), 2-(3,5-Dichloro-1-oxido-pyridine-4-yl)-1-(7-difluoro-methoxy-2′,3′,5′,6′-tetrahydro-spiro[1,3-benzodioxole-2, 4′-(4H)-thiopyran-1′,1′-dioxide]-4-yl)ethanone, also referred to herein as “orismilast”, was disclosed in WO 2011/160632, relating to benzodioxole and benzodioxepene heterocyclic compounds useful as PDE4 inhibitors for use in the treatment, prevention or alleviation of a variety of diseases, such as dermal diseases or conditions, such as proliferative and inflammatory skin disorders, dermatitis, psoriasis, psoriasis vulgaris, atopic dermatitis, seborrheic dermatitis, contact dermatitis, cancer, epidermal inflammation, alopecia, alopecia areata, skin atrophy, steroid induced skin atrophy, skin ageing, photo skin ageing, acne, urticaria, pruritis, and eczema.

The compound of formula (I) should be understood to include any pharmaceutically acceptable form and salts, hydrates or solvate of the compound. The compound may be present in a crystalline or amorphous form. The compound of formula (I) is considered as being a poorly soluble compound. The compound of formula (I) and salts thereof, and methods for synthesizing the compound, are disclosed in WO 2011/160632, WO 2015/197534, WO 2017/103058, and WO 2018/234299.

In any of the aspects of the invention, the compound of formula (I) may be present in a crystalline form. For example, the compound of formula (I) may be the polymorphic Form E of the compound. Form E of the compound of formula (I) is described in WO 2018/234299 and has an XRPD diffractogram pattern substantially as shown in FIG. 1 of WO 2018/234299, which is incorporated herein by reference thereto. Form E is characterised as having a melting endotherm with an onset temperature of about 217° C. to about 219° C. when measured by DSC with a heating rate of 100° C./minute. Form E may be prepared by crystallisation of a compound of formula (I) from a suitable solvent, for example ethanol or acetone.

Orismilast is a selective and efficient inhibitor of PDE4. It has been found that orismilast is a selective inhibitor of PDE4D and PDE4B. In particular, it has been found that orismilast is a selective inhibitor of the PDE4 isoforms PDE4D3 and PDE4B2. In addition, orismilast has been found to potently inhibit the secretion of TNF-α and IL-1β, two cytokines that are highly associated with inflammation. The compound also inhibits IFN-γ. IFN-γ is a T-cell derived Th1 cytokine that plays a role in Th1 immune responses. The ability of orismilast to inhibit cytokines involved in inflammation, particularly those which are linked to neutrophilic dermatoses, such as PG, supports the use of orismilast in the treatment of these diseases. Significantly, orismilast has been shown to an average of 23 times more potent than apremilast on a molar basis, in both LPS and SEB-induced TNF-α secretion from human whole blood.

### Pharmaceutical Compositions

The compound of formula (I), or the pharmaceutically acceptable salt, solvate or hydrate thereof may be comprised within a formulation. Thus, the present invention also provides a formulation, or a composition, comprising the compound of formula (I), or the pharmaceutically acceptable salt, solvate or hydrate thereof, for use in the treatment of neutrophilic dermatoses, such as PG. Compositions and formulations may be prepared according to, for example, the desired mode of administration e.g. oral, topical or intravenous administration.

Compositions and formulations comprising the compound may optionally further comprise one or more viscosity modifying agents, carriers (e.g. mannitol, lactose, microcrystalline cellulose or trehalose), emulsifiers, surfactants, humectants, oils, waxes, polymers, preservatives, pH modifying agents (for example a suitable acid or base, for example an organic acid or organic amine base), buffers, stabilizers, electrolytes antioxidants (for example, butylated hydroxyanisol or butylated hydroxytoluene), crystallisation inhibitors (for example a cellulose derivative such as hydroxypropylmethyl cellulose or polyvinylpyrrolidone), colorants, fragrances and taste-masking agents. Such excipients are well-known, for example as listed in the Handbook of Pharmaceutical Excipients, 7th Edition, Rowe et al.

In some embodiments the compound of formula I, or a pharmaceutically acceptable salt, solvate or hydrate thereof, is present in the formulation in an amount of from about 0.01 to 50% by weight of a solid formulation. Thus it may be that the compound of formula I, or a pharmaceutically acceptable salt, solvate or hydrate thereof, is present in the solid formulation in an amount of about 0.05 to 40%, from 0.1 to 30%, from 0.2 to 20%, from 0.3 to 15%, from 0.4 to 12%, from 0.5 to 11%, from 1 to 10%, from 1.5 to 9.5%, from 2 to 9%, from 2.5 to 8.5%, from 3 to 8%, from 3.5 to 7.5%, from 4 to 7%, from 4.5 to 6.5%, or from about 5 to 6%, e.g. about 5.5%.

### Formulations for Topical Administration

In some embodiments the compound is formulated for topical administration to the subject. For example, a formulation comprising the compound of formula (I), or a pharmaceutically acceptable salt, solvate or hydrate thereof, may be topically applied to the skin at a site affected by neutrophilic dermatoses (e.g. PG), for example by topically applying the formulation directly to a lesion (e.g. an ulcer associated with PG). Formulations suitable for topical administration include liquid or semi-liquid preparations such as liniments, lotions, gels, sprays or foams; oil-in-water or water-in-oil emulsions such as creams, ointments or pastes; or solutions or suspensions such as drops or sprays.

For topical administration, the compound of formula I may typically be present in the formulation in an amount of from 0.01 to 20% by weight of the composition. For example, the compound may be present in the formulation in an amount of from 0.02 to 18%, from 0.03 to 15%, from 0.04 to 15%, from 0.05 to 10%, from 0.06 to 9%, from 0.07 to 8%, from 0.08 to 6%, from 0.09 to 5%, from 0.1 to 4.5%, from 0.15 to 4%, from 0.2 to 3.5%, from 0.3 to 3%, from 0.4 to 2.5%, from 0.5 to 2%, from 0.6 to 1.5% or from 0.7 to 1%, by weight of the composition In some embodiments, the compound may be present in the formulation in an amount of from 0.01 to 5%, or from 0.02 to 2%.

### Formulations for Oral Administration

Formulations comprising the compound of formula (I), or a pharmaceutically acceptable salt, solvate or hydrate thereof, suitable for oral administration may be in the form of discrete units such as capsules, sachets, tablets or lozenges, each containing a predetermined amount of the compound of formula (I). The discrete units may contain the formulation in the form of a powder or granules, a solution or a suspension in aqueous or non-aqueous liquid, such as ethanol or glycerol, or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose, hydroxypropyl methylcellulose, hydroxypropylcellulose, carbomers and polyvinylpyrrolidone. The formulation may also be administered in the form of a bolus, electuary or paste.

Powders may be prepared using well-known methods, for example by milling, blending, micro-precipitation, lyophilisation or spray drying, or spray-freeze drying a solution comprising a compound of formula (I), or a pharmaceutically acceptable salt, solvate or hydrate thereof.

The amount of the compound of formula (I), or a pharmaceutically acceptable salt, solvate or hydrate thereof, in each oral dosage form (e.g. tablet, capsule, sachet or lozenge) may range from about 1 mg to about 100 mg. The amount of the compound may for example range from 5 mg to 80 mg, from 10 mg to 60 mg, from 15 mg to 50 mg, from 20 to 40 mg or from 25 to 30 mg. In some embodiments, the amount of the compound of formula I, or a pharmaceutically acceptable salt, solvate or hydrate thereof, in each oral dosage form (e.g. tablet, capsule, sachet or lozenge) may range from about 10 mg to about 40 mg, for example from about 10 to about 30 mg. In some embodiments, the amount of the compound of formula I, or a pharmaceutically acceptable salt, solvate or hydrate thereof, in each oral dosage form (e.g. tablet, capsule, sachet or lozenge) is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg.

In certain embodiments particles comprising the compound of formula (I), or a pharmaceutically acceptable salt, solvate or hydrate thereof, may be prepared by precipitation, lyophilisation or spray drying, or spray-freeze drying a solution comprising the compound and a suitable carrier to provide powder particles comprising the compound of formula (I) and the carrier as composite particles. Suitable carriers include inert carriers such as starch, sugars (e.g. mannitol, lactose, microcrystalline cellulose or trehalose).

In some embodiments the compound of formula (I), or a pharmaceutically acceptable salt, solvate or hydrate thereof is present in the formulation as a micronised powder, for example a micronised crystalline powder. Thus it may be that the particle size distribution of the compound in the formulation has a D50≤25 μm, for example D50≤20 μm, D50≤10 μm, D50:5 5 μm, or D50:S 3 μm.

Powders comprising a compound of formula (I), or a pharmaceutically acceptable salt, solvate or hydrate thereof, as described herein, may be dissolved or suspended in a suitable solvent (preferably water) prior to administration, e.g. application of a spray or gel.

A tablet may be made by compressing or moulding the formulation, optionally with one or more accessory ingredients. Compressed tablets may be prepared by compressing, in a suitable machine, the formulation in a free-flowing form such as a powder or granules, optionally mixed by a binder, such as e.g. lactose, glucose, starch, gelatine, acacia gum, tragacanth gum, sodium alginate, carboxymethylcellulose, methylcellulose, hydroxypropyl methylcellulose, polyethylene glycol, waxes or the like; a lubricant such as sodium oleate, sodium stearate, magnesium stearate, sodium benzoate, sodium acetate, sodium chloride or the like; a disintegrating agent such as starch, methylcellulose, agar, bentonite, croscarmellose sodium, sodium starch glycollate, crospovidone or the like or a dispersing agent, such as polysorbate 80.

Moulded tablets may be made by moulding. Suitable techniques for moulding tablets are well-known in the art. For example, in a suitable machine, a mixture of the powdered active ingredient and suitable carrier may be moistened with an inert liquid diluent. In some embodiments, moulded tablets may be made by dispersing a water-soluble excipient with the powdered active ingredient in a suitable solvent such as water, alcohol or organic solvents (e.g. acetone, hydrocarbons). Alternatively, moulded tablets may be made using thermoplastic polymers (e.g. polyethyleneoxide or polyvinyl caprolactam-polyvinylacetate-polyethylene glycol copolymers), without an inert liquid diluent.

### Modified Release Formulation

In some embodiments, the compound of formula (1), or a pharmaceutically acceptable salt, solvate or hydrate thereof, is comprised within a modified release formulation, for example a modified release formulation for oral administration. Thus it may be that the compound may be comprised within a modified release tablet formulation for oral administration. The use of modified release formulations may control the release of the therapeutic agent and thus control drug absorption from gastrointestinal tract. It has previously been described by the Applicant (in a PCT application published as WO2020/148271) that beneficial effects with respect to improved tolerability towards gastrointestinal adverse events and maintained systemic exposure can be achieved by formulating a PDE4 inhibitor in a modified release tablet formulation, wherein the in-vitro release is fast in comparison to a typically modified release profile but not yet as fast as for an immediate release tablet where major tolerability issues were seen.

It will be appreciated that the rate of dissolution of a modified release formulation will be determined by several factors e.g. the type and quantity of hydrophilic matrix former, excipients (fillers and coating) and the particle size of the drug substance. The dissolution target area in FIG. 1 illustrates the optimal area. Preferably, the modified release formulation provides the release and dissolution of the compound of formula (I) within the optimal area. The dissolution profile of a given formulation can be determined using the methods described in WO2020/0148271.

In some embodiments the orismilast is formulated as a modified release formulation described in WO2020/0148271, which is incorporated herein by reference thereto.

In some embodiments the modified release formulation releases less than 40% of the compound of formula (I) after 12 minutes. In some embodiments the modified release formulation releases less than 40% of the compound of formula (I) after 30 minutes. In some embodiments the modified release formulation releases less than 35% of the compound of formula (I) after 30 minutes. In some embodiments the modified release formulation releases from about 20% to about 40% of the compound of formula (I) after 30 minutes. In some embodiments the modified release formulation releases from about 25% to about 35% of the compound of formula (I) after 30 minutes. In some embodiments the modified release formulation releases from about 11% to about 65% of the compound of formula (I) after 45 minutes. In some embodiments the modified release formulation releases from about 25% to about 60% of the compound of formula (I) after 45 minutes. In some embodiments the modified release formulation releases from about 30% to about 45% of the compound of formula (I) after 45 minutes. In some embodiments the modified release formulation releases from about 35% to about 55% of the compound of formula (I) after 60 minutes. In some embodiments the modified release formulation releases more than about 60% of the compound of formula (I) after 60 minutes. In some embodiments the modified release formulation releases from about 35% to about 50% of the compound of formula (I) after 60 minutes. In some embodiments the modified release formulation releases from about 40% to about 50% of the compound of formula (I) after 60 minutes. In some embodiments the modified release formulation releases from about 50% to about 60% of the compound of formula (I) after 90 minutes. In some embodiments the modified release formulation releases from about 60% to about 80% of the compound of formula (I) after 120 minutes. In some embodiments the modified release formulation releases from about 60% to about 70% of the compound of formula (I) after 120 minutes. In some embodiments the modified release formulation releases more than about 70% of the compound of formula (I) after 180 minutes. In some embodiments the modified release formulation releases more than about 80% of the compound of formula (I) after 180 minutes. In some embodiments the modified release formulation releases from about 70% to about 100% of the compound of formula (I) after 180 minutes. In some embodiments the modified release formulation releases from about 80% to about 100% of the compound of formula (I) after 180 minutes. In some embodiments the modified release formulation releases from about 85% to about 100% of the compound of formula (I) after 180 minutes. In some embodiments the modified release formulation releases from about 90% to about 100% of the compound of formula (I) after 180 minutes. In some embodiments the modified release formulation releases from about 95% to about 100% of the compound of formula (I) after 180 minutes. In certain embodiments the modified release formulation releases from about 11% to about 65% of the compound of formula (I) after 45 minutes and more than 80% of the compound of formula (I) after 180 minutes. In certain embodiments the modified release formulation releases from about 25% to about 65% of the compound of formula (I) after 45 minutes and at least 75% of the compound of formula (I) after 180 minutes. In certain embodiments the modified release formulation releases from about 30% to about 50% of the compound of formula (I) after 45 minutes and at least 75% of the compound of formula (I) after 180 minutes. In some embodiments the modified release formulation releases from about 30% to about 55% of the compound of formula (I) after 45 minutes and at least 85% of the compound of formula (I) after 180 minutes. In certain embodiments the modified release formulation releases from about 30% to about 50% of the compound of formula (I) after 45 minutes and about 80% to about 100% of the compound of formula (I) after 180 minutes. In some embodiments the modified release formulation releases about 50% of the compound of formula (I) between about 60 and 100 minutes. In some embodiments the modified release formulation releases about 80% of the compound of formula (I) between about 120 and 180 minutes. In some embodiments the modified release formulation releases less than 40% of the compound of formula (I) after 30 minutes; 50% of the compound of formula (I) is released between 60 and 100 minutes; and 80% of the compound of formula (I) is released between 115 and 180 minutes. The modified release composition may, for example, be any of the modified release compositions described herein. In some embodiments the modified release composition comprises a compound of the formula (I), or a pharmaceutically acceptable salt, solvate or hydrate thereof; and a pharmaceutically acceptable hydrophilic matrix former (e.g. HPMC). In some embodiments the modified release composition comprises a compound of the formula (I), or a pharmaceutically acceptable salt, solvate or hydrate thereof; a pharmaceutically acceptable hydrophilic matrix former (e.g. HPMC); and a filler (e.g. lactose monohydrate). In some embodiments the modified release composition comprises a compound of the formula (I), or a pharmaceutically acceptable salt, solvate or hydrate thereof; and 15% w/w to 30% w/w of a pharmaceutically acceptable hydrophilic matrix former (e.g. HPMC). In some embodiments the modified release composition comprises a compound of the formula (I), or a pharmaceutically acceptable salt, solvate or hydrate thereof; and 15% w/w to 25% w/w of HPMC.

In the paragraph above reference to “release of the compound of formula (I)” refers to the % by weight of the compound of formula (I) initially present in the modified release formulation that is released into a dissolution medium at the specified time point, wherein the dissolution is determined using Ph. Eur. 2.9.3 Apparatus II, with a dissolution medium of 900 ml 0.5% sodium dodecyl sulfate in 0.1N HCl and a paddle speed of 75 rpm. The amount of the compound of formula (I) present in the dissolution medium may be determined by reversed phase, isocratic HPLC using a C18 column and UV detection at 272 nm. Suitably the % release values of the compound of formula (I) is a mean value obtained by measuring the release profile of more than one sample of the modified release formulation, thereby reducing the effects of inter- or intra-batch variability. The mean release % may be obtained by measuring the release from, for example 6, 12 or 24 samples of the modified-release formulation.

In some embodiments, the modified release tablet formulation comprises:
(i) the compound of formula (1), or a pharmaceutically acceptable salt, solvate or hydrate thereof;
(ii) one or more of a pharmaceutically acceptable hydrophilic matrix former;
(iii) optionally, one or more pharmaceutically acceptable excipients selected from the group consisting fillers, glidants and lubricants; and
(iv) optionally a pharmaceutically acceptable coating system.

In some embodiments the hydrophilic matrix former in the modified release formulation comprises hydroxypropyl methylcellulose or hydroxypropylcellulose, or mixtures thereof.

In some embodiments the hydrophilic matrix former in the modified release formulation comprises hydroxypropyl methylcellulose (HPMC). In some embodiments the hydrophilic matrix former in the modified release formulation consists of HPMC. In some embodiments the HPMC has a viscosity of from 30 to 150 mPa·s. In some embodiments the HPMC has a viscosity of from 35 to 130 mPa·s. In some embodiments the HPMC has a viscosity of from 40 to 60 mPa·s. In some embodiments the HPMC has a viscosity of from 80 to 120 mPa·s. Reference herein to the viscosity of HPMC refers to the viscosity of a 2% (w/w) solution of the HPMC in water at 20° C. in accordance with United States Pharmacopoeia (USP XXIII).

In some embodiments the hydrophilic matrix former in the modified release formulation comprises HPMC with a methoxyl substitution of from about 5% to about 35% In some embodiments the HPMC has a methoxyl substitution of from about 15% to about 30%. In some embodiments the HPMC has a methoxyl substitution of from about 19% to about 24%. In some embodiments the HPMC has a methoxyl substitution of from about 25% to about 35%. In some embodiments the HPMC has a methoxyl substitution of from about 28% to about 30%.

In some embodiments the hydrophilic matrix former in the modified release formulation comprises HPMC with a hydroxypropyl substitution of from about 4% to about 15%. In some embodiments the hydrophilic matrix former in the modified release formulation comprises HPMC with a hydroxypropyl substitution of from about 4% to about 12%. In some embodiments the hydrophilic matrix former in the modified release formulation comprises HPMC with a hydroxypropyl substitution of from about 7% to about 12%.

In some embodiments the hydrophilic matrix former in the modified release formulation comprises HPMC with a methoxyl substitution of from about 19% to about 24%; and a hydroxypropyl substitution of from about 4% to about 12%.

In some embodiments the hydrophilic matrix former in the modified release formulation comprises HPMC with a methoxyl substitution of from about 19% to about 24%; and a hydroxypropyl substitution of from about 7% to about 12%.

In some embodiments the hydrophilic matrix former in the modified release formulation comprises HPMC with a methoxyl substitution of from about 28% to about 30%; and a hydroxypropyl substitution of from about 7% to about 12%.

In some embodiments the hydroxypropyl methylcellulose is Hypromellose 2910, hypromellose 2208, or mixtures thereof.

Suitably the hydrophilic matrix former is present in a concentration from about 10% w/w to about 30% w/w hydroxypropyl methylcellulose. Thus it may be that the hydrophilic matrix former is present in a concentration from about 15% w/w to about 25% w/w hydroxypropyl methylcellulose. For example, wherein the hydrophilic matrix former is present in a concentration of 17.5% w/w hydroxypropyl methylcellulose. The hydroxypropyl methylcellulose may be, for example, any of the grades of hydroxypropyl methylcellulose disclosed herein (e.g. Hypromellose 2910, Hypromellose 2208, or mixtures thereof).

In some embodiments the one or more pharmaceutically acceptable excipients present in the modified release formulation comprises a filler, selected from lactose monohydrate and microcrystalline cellulose, and mixtures thereof. the fillers are present in a concentration from about 30% to about 78% w/w of lactose monohydrate and from 0% to about 40% w/w of microcrystalline cellulose. In some embodiments the filler is lactose monohydrate. In some embodiments the filler is lactose monohydrate and is present in a concentration from about 30% to about 78% w/w. Thus it may be that the lactose monohydrate is present in a concentration of about 71% w/w.

In some embodiments the modified release formulation comprises a coating. For example a PVA-based coating system.

In some embodiments the modified release formulation comprises
(i) the compound of formula (I), or a pharmaceutically acceptable salt, solvate or hydrate thereof;
(ii) a hydrophilic matrix former, wherein the hydrophilic matrix former is present in a concentration of from about 15% w/w to about 25% w/w hydroxypropyl methylcellulose;
(iii) from about 30% w/w to about 78% w/w lactose monohydrate;
(iv) optionally one or more pharmaceutically acceptable excipients selected from the group consisting of glidants and lubricants; and
(v) optionally a pharmaceutically acceptable coating system.

In some embodiments the modified release formulation comprises the compound of formula (I), about 0.5% w/w colloidal silicon dioxide, about 1.0% w/w magnesium stearate; and optionally a PVA-based coating system.

In some embodiments the modified release formulation comprises the compound of formula (I), about 17.5% w/w hypromellose, about 71.0% w/w lactose monohydrate, about 0.5% w/w colloidal silicon dioxide, about 1.0% w/w magnesium stearate; and optionally a PVA-based coating system.

In some embodiments the compound of formula (I) is present in the modified release formulation in an amount of from about 1% w/w to about 40% w/w, for example about 1% w/w to about 30% w/w, from about 1% w/w to about 20% w/w, or from about 2% w/w to about 15% w/w. In some embodiments the compound of formula (I) is present in the modified release formulation in an amount of from about 2% w/w to about 5% w/w. In some embodiments the compound of formula (I) is present in the modified release formulation in an amount of from about 8% w/w to about 12% w/w.

In some embodiments the compound of formula (I) is present in the modified release formulation in an amount of from about 5 mg to about 60 mg; about 10 mg to about 50 mg. For example about 10 mg, or about 30 mg.

In some embodiments, the compound may be evenly distributed in the modified release tablet formulation. The compound may be micronized. In some embodiments, the compound is crystalline. In some embodiments, the compound is crystalline and micronized.

In some embodiments the formulation comprises polymorphic form E of the compound of formula (I). In some embodiments, the polymorphic form E of the compound is micronized. In some embodiments, the polymorphic form E of the compound is crystalline and micronized.

The hydrophilic matrix former may be hydroxypropyl methylcellulose (HPMC), hydroxypropylcellulose (HPC), or mixtures thereof. For example, the hydrophilic matrix former could be hydroxypropyl methylcellulose, and mixtures thereof. The hydrophilic matrix former may be present at various concentrations and combinations from about 10% w/w to about 30% w/w HPMC. In some embodiments the hydrophilic matrix former is present in an amount of from about 15% w/w to about 25% w/w HPMC. In some embodiments the hydrophilic matrix former is present in an amount of from about 15% w/w to about 20% w/w HPMC. In some embodiments the hydrophilic matrix former is present in an amount of 17.5% w/w HMPC.

In some embodiments the modified release tablet formulation comprises one or more fillers and/or binders. The term “filler” as used herein may also function as a binder. The filler or binder may be selected from lactose monohydrate, lactose hydrous or anhydrous, microcrystalline cellulose, mannitol, isomalt, and mixtures thereof. For example, the filler could be lactose monohydrate. The filler may be present at various concentrations from about 30% w/w to about 78% w/w. For example, the filler may comprise about from about 30% w/w to about 78% w/w of lactose monohydrate and from about 0 to about 40% w/w of microcrystalline cellulose. For example, the filler could be lactose monohydrate in an amount of about 71% w/w.

In some embodiment the modified release tablet formulation comprises one or more glidants. The term “glidant” as used herein includes colloidal silicon dioxide, talc, etc. For example, the glidant could be colloidal silicon dioxide. The glidant may be present at various concentrations from about 0.1% w/w to about 2% w/w, for example from about 0.2% w/w to about 1% w/w, e.g. about 0.5% w/w.

In some embodiment the modified release tablet formulation further comprises one or more lubricants. The term “lubricant” as used herein includes magnesium stearate, sodium stearyl fumarate, talc, etc. For example, the lubricant may be magnesium stearate. The lubricant may be present at various concentrations from about 0.1% w/w to about 2% w/w, for example from about 0.5% w/w to about 1.5% w/w, e.g. about 1.0% w/w.

Suitably the % w/w of the components comprising the modified release tables (e.g. the compound of formula (I), the hydrophilic matrix former, the filler, glidant and/or lubricant) refer to the % w/w prior to adding the optional coating to the tablet. Thus as will be realised by the skilled person, in those embodiments where the modified release tablets are coated the % w/w of the components in the core of the tablet will be lower that those in the uncoated tablet cores due to the additional weight of the coating.

In some embodiments the modified release tablet formulation may comprise a film coating of the tablet cores. A suitable coating may be a PVA-based coating system. The term “coating system”, as used herein includes HPMC-based coating systems, PVA-based coating systems (polyvinyl alcohol), PVA-PEG based coating systems (polyethylene glycol) or ethylcellulose based functional barrier membrane coating systems. For example, the coating system could be the PVA-based coating system. For example, the coating system could be Opadry® II. For example the coating system could be present in an amount from about 3% to about 5% weight gain of the tablet formulation, for example a 4% weight gain.

In some embodiments, the particle size distribution of the compound in the tablet formulation may be D50≤25 μm, for example D50≤20 μm, D50≤10 μm, D50≤5 μm, or D50≤3 μm.

The amount of the compound of formula (1) in each tablet may range from about 1 mg to about 100 mg, or from about 5 mg to about 60 mg. The amount of the compound may for example range from 10 mg to 50 mg, from 20 mg to 45 mg, and from 30 mg to 40 mg. In some embodiments, the amount of the compound in each tablet may be from about 10 to about 30 mg. In some embodiments, the amount of the compound of formula (I) in each tablet is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg.

In some embodiments, the compound of formula (1), or a pharmaceutically acceptable salt, solvate or hydrate thereof, is comprised within a granulated blend formulation. A granulated blend formulation may comprise:
the compound of formula (1), or a pharmaceutically acceptable salt, solvate or hydrate thereof; and
one or more of a pharmaceutically acceptable hydrophilic matrix former;
one or more pharmaceutically acceptable excipients selected from the group consisting fillers, binders, glidants and lubricants; and
a hard capsule shell material.

The hydrophilic matrix former could be hydroxypropyl methylcellulose (HPMC), hydroxypropylcellulose (HPC), or mixtures thereof. The hydrophilic matrix former could be present at various concentrations and combinations from about 10% w/w to about 20% w/w HPMC.

The fillers/binders could be selected from lactose monohydrate, lactose hydrous or microcrystalline cellulose, and mixtures thereof. The fillers/binders could be present at various concentrations from about 20% w/w to about 75% w/w of lactose monohydrate and from 0 to about 50% w/w of microcrystalline cellulose. The glidant could be colloidal silicon dioxide, which could be present at various concentrations from about 0.1% w/w to about 2% w/w.

The lubricant could be magnesium stearate, which could be present at various concentrations from about 0.1% w/w to about 2% w/w.

In some embodiments the compound of formula (I) is present in the granulated blend formulation in an amount of from about 1% w/w to about 40% w/w, for example about 1% w/w to about 30% w/w, from about 1% w/w to about 20% w/w, or from about 2% w/w to about 15% w/w.

The blend formulation could be dispensed in a hard capsule. Capsule shell material for hard capsules could be made of several materials such as gelatin (pig, bovine, fish etc), hydroxypropyl methylcellulose (HPMC), polyvinyl alcohol, starch and pullulan could be applied.

In some embodiments the granulated blend formulation is formulated as a unit dosage form (e.g. a capsule formulation). The amount of the compound of formula (1) in each unit dose form may range from about 1 mg to about 100 mg, or from about 5 mg to about 60 mg. The amount of the compound may for example range from 10 mg to 50 mg, from 20 mg to 45 mg, and from 30 mg to 40 mg. In some embodiments, the amount of the compound in unit dosage form comprising the granulated blend formulation may be from about 10 to about 30 mg. In some embodiments, the amount of the compound of formula (I) in each unit dosage form is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg

A manufacturing process for the granulated blend formulation could consist of blending and sieving steps of the drug substance and excipients followed by granulation, e.g. roller compaction, and encapsulation.

In another aspect, the present invention relates to a method of treating neutrophilic dermatoses, such as PG, the method comprising administering to a subject in need thereof a modified release tablet formulation comprising the compound of formula (I), or a pharmaceutically acceptable salt, solvate or hydrate thereof.

### Formulations for Parenteral Administration

Formulations of the present invention suitable for parenteral (e.g. intravenous, intramuscular or subcutaneous) administration may be in the form of granules, powder or a concentrated liquid (e.g. a solution or suspension) which can be reconstituted or diluted prior to use by the addition of a liquid, such as an aqueous liquid (e.g. water or saline) to form an essentially clear, stable liquid comprising the formulation dissolved in solution. The reconstituted or diluted solution also forms part of the present invention.

### Treatment of Neutrophilic Dermatoses

The present invention provides a compound of formula (I), or a pharmaceutically acceptable salt, solvate or hydrate thereof, for use in the treatment of neutrophilic dermatoses, such as PG. Also provided is a method for treating neutrophilic dermatoses, such as PG, the method comprising administering to a subject in need thereof a therapeutically effective amount of a compound of formula (I), or a pharmaceutically acceptable salt, solvate or hydrate thereof.

Neutrophilic dermatoses are a heterogenous group of non-infectious anti-inflammatory diseases, including PG, which are characterised by infiltration of the epidermis, dermis and/or hypodermis by neutrophils. While the pathogenesis of PG remains unclear, upregulation of a number of key proinflammatory and neutrophil chemotactic factors within lesional skin have been identified, including IL-1β, IL-17, TNFα, IL-8, IL-6, IL-23, MMP9 and MMP10 (George et al., Clinical Medicine Journal, May 2019). Expression of IL-23, which plays a critical role in driving inflammation associated with IL-17 production and especially neutrophil recruitment, has been shown to be highly elevated at both a transcriptional and protein level in a recalcitrant PG lesion (Guenova et al, Arch Dermato, 2011 Oct;147(10):1203-5). IL-8 has been demonstrated to produce PG in animal models (Oka et al., Lab. Invest. 2000; 80: 595-604). A significant increase in interleukin (IL)-1P and in the IL-1p receptor in skin samples of PG patients has also been reported (Galimberti et al., JAAD Case Rep. 2016 September; 2(5): 366-368), In a number of studies, patients suffering from PG have been successfully treated with anti-TNFα antibodies (Brooklyn et al., Gut. 2006 April; 55(4): 505-509; Ljung et al., Scand J Gastroenterol, 2002 Sep;37(9):1108-10; Tan et al., Arch Dermatol. 2001 July; 137(7):930-3).

PDE4 is an enzyme that reduces levels of intracellular cyclic adenosine monophosphate (cAMP), a pro-inflammatory molecule that stimulates production of cytokines including TNF-α, IL-17 and IL-23. PDE4s are the predominant cAMP-degrading isozymes in most, if not all, immune and inflammatory cells, including T cells, B cells, eosinophils, neutrophils, dendritic cells, monocytes, and macrophages (Torphy, Am J Respir Crit Care Med 1998; 157:351-70). Three PDE4 subtypes, PDE4A, PDE4B and PDE4D, are expressed in these cells, while PDE4C is minimal or absent (Press et al., Prog Med Chem 2009; 47:37-74). It has been demonstrated that the pharmacological effects of PDE4 inhibitors on macrophage TNF-α production are mediated exclusively through inhibition of PDE4B (Jin et al., J Immunol 2005;175: 1523-31; Jin et al., Proc Natl Acad Sci USA 2002;99: 7628-33). PDE4D is a predominant subtype conducting “long-term” lymphocyte responses, such as IFN-γ and IL-5 release and proliferation (Peter et al., J Immunol 2007;178: 4820-31). It has also been shown that ablation of PDE4D or PDE4B, but not PDE4A, has profound effects on neutrophil functions (Ariga et al., J Immunol 2004; 173:7531-8).

The side effects associated with PDE4 inhibitors are thought to be a result of their non-selectivity to all four PDE4 subtypes, and thus generation of new PDE4 inhibitors with subtype selectivity may provide clinical benefits by maintaining therapeutic efficacy while decreasing the side effects (Manning et al., Br J Pharmacol 1999;128: 1393-8). This notion is supported by a series of studies in which PDE4 gene-targeted mice were used to define the function of individual PDE4 subtypes (Jin et al., Cyclic Nucleotide Phosphodiesterases in Health and Disease Boca Raton, FL: CRC Press, 2007:323-46).

In vitro pharmacology studies have demonstrated that the compound of formula (I) is a potent and selective PDE4 inhibitor, especially of the PDE4B and PDE4D subtypes. The compound has also been found to inhibit the secretion of tumour necrosis factor-alpha (TNF-α), interferon gamma (IFN-γ) and interleukin (IL)-1, -2 and -4. Furthermore, both oral and topical administration of the compound was shown to exhibit anti-inflammatory effects in a mouse model. These characteristics make the compound of formula (I) particularly suitable for the treatment of PG and other neutrophilic dermatoses. Without being bound by theory, it is believed that the ability of the compound of formula (I) to specifically inhibit the PDE4 isoforms PDE4B and PDE4D, which are related to inflammation, may provide an improved therapeutic window compared with other PDE4 inhibitors, such as apremilast and roflumilast which are known to be broad unspecific inhibitors of PDE4. Subtype specificity may be of importance as recent research indicates that the order of importance for anti-inflammatory effects appears to be inhibition of PDE4B>PDE4D>PDE4A, with no or very limited beneficial effects from PDE4C inhibition. Furthermore, as PDE4B2 expression in the brain is low, this from a theoretical standpoint is a good target to reduce potential systemically driven adverse events. The compound of formula (I) thus offers a more optical approach to PDE4 inhibition which balances anti-inflammatory effects and tolerability, in particular reducing unwanted side effects associated with treatment, thereby providing an advantage over known PDE4 inhibitors.

In some embodiments, the compound of the invention is for use in treating a symptom of neutrophilic dermatoses, such as PG. For example, the compound may be for use in eliminating, promoting healing of, or reducing the severity, spread, size, depth, growth rate and/or number of, inflammatory nodules, blisters, sores, pustules, abscesses, comedones, ulcers and/or sinus tracts associated with neutrophilic dermatoses, such as PG. In some embodiments the compound of the invention is for use in eliminating, promoting healing of, or reducing the size, depth, growth rate, number or spread of ulcers or sores associated with PG.

In some embodiments, the compound of the invention is for use in promoting (e.g. increasing the rate of) the healing of ulcers or sores associated with PG. In some embodiments, the compound of the invention is for use in preventing the development of additional ulcers or sores associated with neutrophilic dermatoses, such as PG.

In some embodiments, the compound is for use in reducing inflammation caused by or associated with neutrophilic dermatoses, such as PG. In some embodiments the compound is for use in reducing inflammation caused by or associated with PG, wherein the compound reduces one or more inflammatory biomarkers associated with PG in the subject. For example the compound of the invention may reduce one or more of: IL-1p and/or its receptors I and/or II, IL-8, C—X—C motif ligand (CXCL), CXCL 16, RANTES (regulated on activation, normal T cell expressed and secreted), Fas, Fas ligand, CD40, CD40 ligand, TNFα, IL-6, IL-17, IL-23 and IL-36a. In some embodiments, the compound reduces the one or more inflammatory biomarkers in lesional skin. In some embodiments, the compound is for use in reducing infiltrating neutrophils in lesional skin.

In some embodiments, the compound is for use in eliminating or reducing scarring caused by or associated with neutrophilic dermatoses, such as PG.

In some embodiments, the compound is for use in reducing pain caused by or associated with neutrophilic dermatoses, such as PG. Pain may be assessed according to the Patient's Global Assessment of Skin Pain (0=no pain, 10=worst imaginable pain) 0-10 numerical rating scale (NRS) (Newton et al., J Patient Rep Outcomes. 2019;3(1):42.). Suitably, NRS is reduced by at least 30%, compared to baseline. Other well-known pain scoring systems can be used to assess the reduction in pain associated with the neutrophilic dermatoses, such as PG. For example the reduction of pain could also be assessed using the Visual analog scale of pain (VAS pain), which also assesses pain on a visual scale of 0 (no pain) to 10 (worst imaginable pain).

Pain may also be assessed according to the McGill Pain questionnaire. The McGill Pain questionnaire can be used to evaluate the sensation, strength and change over time of experienced pain. It can monitor pain over time or determine the effectiveness of intervention (Melzack, Pain: September 1975, Volume 1, Issue 3, p277-299).

In some embodiments the pain associated with the neutrophilic dermatoses, such as PG, is reduced by at least about 10%, 20%, 30%, 40%, 50%, 60%, 70%, 80% or 90% using a suitable pain scoring method (e.g. one of the scoring methods described herein) relative to the baseline pain level prior to treatment with the compound of formula (I).

In some embodiments, the Dermatology Life Quality Index (DLQI) of the patient treated with the compound is reduced during the treatment period. In some embodiments the DLQI of the subject is reduced by at least about 10%, 20%, 30%, 40%, 50%, 60%, 70%, 80% or 90% relative to the baseline level prior to treating the subject with the compound. In some embodiments, the DLQI of the patient is reduced by at least 50%, such as at least 75% or such as at least 90%.

DLQI is a questionnaire of 10 questions concerning the patients' perception of the impact of skin diseases on different aspects of their health-related quality of life over the last week. Each question is scored on a four-point scale (0-3) resulting in a range of 0-30 points (0=Disease has no impact on quality of life, 30=Disease has maximum impact on quality of life). A validated scale first introduced by Finlay and Khan, Clin. Exp. Dermatol., 19 (1994), pp. 210-216).

In some embodiments, the Work Productivity and Activity Questionnaire (WPAI) impairment percentage of the patient treated with the compound is reduced during the treatment period. Suitably, the impairment score of the patient is reduced by at least 50%, such as at least 75% or such as at least 90%.

WAPI is a questionnaire describing work impairment due to a specific disease. Outcomes are expressed as impairment percentages, with higher numbers indicating greater impairment and less productivity (Reilly et al., Pharmacoeconomics. 1993 November; 4(5):353-65).

In some embodiments, the Anxiety and Depression (HADS) score of the patient treated with the compound is reduced during the treatment period. Suitably, the HADS score of the patient is reduced by at least 50%, such as at least 75% or such as at least 90%.

HADS is a questionnaire comprising seven questions for anxiety and seven questions for depression. Each question is connected to four answers retrieving 0-3 points. For each condition, 0-7 points corresponds to normal case; 8-10 to borderline abnormal; and 11-21 to abnormal case (Zigmond and Snaith, Acta Psychiatrica Scandinavica (1983), 67(6): 361-370).

In some embodiments, the European quality of life—5 Dimensions (EQ-5D) score of the patient treated with the compound is increased during the treatment period. Suitably, the EQ-5D score of the patient is increased by at least 50%, such as at least 75% or such as at least 90%.

EQ-5D is a standardized instrument for measuring generic health status in terms of five dimensions: mobility, self-care, usual activities, pain/discomfort, and anxiety/depression. Each dimension receives a value of 1-5 leaving 55 55) different health states. The score is combined with an overall patient rating of health from 0-100 where 0 is worst imaginable health and 100 is best imaginable health. The scale was further developed from the original EQ-5D by Herdman et al., Qual Life Res. 2011 December; 20(10):1727-36.

In some embodiments, the Multidimensional Fatigue Inventory 20 (MFI-20) response of the patient treated with the compound is improved during the treatment period.

MFI-20 was invented by Smets et al., J Psychosom Res 1995; 39: 315-25. It consists of 20 items describing five subscales of fatigue: General Fatigue (GF), Physical Fatigue (PF), Reduced Motivation (RM), Reduced Activity (RA), and Mental Fatigue (MF). For each of the items the respondent must specify the extent to which the particular statements relate to him/her on a five-point scale, ranging from Yes, that is true to No, that is not true.

In a further aspect, the invention provides a method of treating a subject with neutrophilic dermatoses, such as PG, the method comprising administering to the subject a compound of formula (I), or a pharmaceutically acceptable salt, solvate or hydrate thereof. The method may comprise reducing inflammation associated with neutrophilic dermatoses, such as PG.

In another aspect, the invention provides the use of a compound of formula (I), or a pharmaceutically acceptable salt, solvate or hydrate thereof, for the manufacture of a medicament for use in the treatment of neutrophilic dermatoses, such as PG.

In some embodiments the pyoderma gangrenosum (PG) is selected from classic PG, atypical/bullous PG, pustular PG, vegetative PG, malignant PG, peristomal PG and post-operative PG.

In some embodiments the subject has concomitant Hidradenitis suppurativa (HS). HS is an inflammatory disorder of the follicular epithelium and commonly occurs in the axillae, inframammary folds, and groin. HS typically presents with inflammatory nodules, abscesses, comedones, sinus tracts, or scarring. It has an insidious onset, starting with mild discomfort, erythema, burning, pruritus, and hyperhidrosis. It progresses to form tender or deep-seated nodules, which expand and coalesce to form large painful abscesses. The rupture of these abscesses releases malodorous and purulent discharge. Recurrent or persistent HS results in the formation of double-ended comedones, sinus tracts, and scarring. Secondary bacterial infection can often occur.

It has been reported that the prevalence of PG among patients with HS ranges from 0.2% to 0.4% as compared to only 0.01% in patients without a diagnosis of HS. This represents a prevalence of PG which is 18 times higher in HS patients (Malviya and Garg, “A Comprehensive Guide to Hidradenitis Suppurativa”, Chapter 8, 2022). In patients with HS, it has been found that PG can appear at any point after the development of HS and often has a severe, refractory course. (Hsiao et al., Arch. Dermatol. 2010; 146(11):1265-1270).

In some embodiments the subject has PASH syndrome. PASH is a term used to describe the clinical triad of PG, acne and HS in a single patient (Marzano et al., Medicine 93(27):e187; Niv et al., JAAD Case Reports 2017; 3:70-3).

In some embodiments the subject has PASS syndrome (PG, acne conglobate, HS, seropositive spondyloarthropathies), PAPASH syndrome (PG, pyogenic arthritis, acne, HS), or PsAPASH syndrome (psoriatic arthritis, PG, acne, HS).

In some embodiments the subject is suffering from a comorbidity selected from obesity, metabolic syndrome, inflammatory bowel disease (such as Crohn's disease or ulcerative colitis), spondyloarthropathy, inflammatory arthritis, a haematological malignancy (such as leukaemia (e.g. acute myeloid leukaemia) or IgA monoclonal gammopathy), hepatitis (e.g. hepatitis C), blood dyscrasia, granulomatosis with polyangiitis, psoriasis, atopic dermatitis or any combination thereof.

It may be that the subject has not been previously treated with antibody therapy. Additionally or alternatively, it may be that the subject has not been previously treated with a TNF-α inhibitor. It may be that the subject has not been previously treated with a biological therapy (e.g. a biological therapy for PG).

In a further embodiment, the subject has been previously been treated with a biological therapy (e.g. a biological therapy for PG). It may be that the subject is non-responsive or refractory to treatment with a biological therapy (e.g. a biological therapy for PG).

In another embodiment the subject is non-responsive or refractory to one or more therapies other than the compound of formula (I). In some embodiments the subject is non-responsive or refractory to one or more therapies selected from an antibiotic (e.g. dapsone, doxycycline, clindamycin, rifampin or a carbapenem (e.g. ertapenem)), an immunosuppressant (e.g. cyclosporin, tacrolimus, methotrexate), an anti-inflammatory (e.g. colchicine), a steroid (e.g. prednisone) and a biological therapy (e.g. any of the biological therapies for PG disclosed herein, such as an anti-TNFα agent).

Reference herein to a “biological therapy for PG” includes anti-TNF-α biologics (e.g. adalimumab, certolizumab infliximab, etanercept, or golimumab); anti-IL-17 biologics (e.g. bimekizumab, brodalumab, CJM112, ixekizumab or secukinumab); anti-IL-12/23 biologics (e.g. ustekinumab), anti-IL-23 biologics (e.g. guselkumab, risankizumab, ortildrakizumab); an anti-IL-1 biologic (e.g. anakinra, bermkimab or canakinumab); an anti CD (e.g. iscalimab); or an anti-IL-36 biologic (e.g. spesolimab or ismidolimab); anti CXCR1/CXCR2 biologics (e.g. LY 3041658), or a Complement C5a inhibitor, or any combination thereof. In some embodiments the biological therapy for PG is an anti-TNF-α biologic (e.g. adalimumab, infliximab, golimumab or certolizumab pegol). In some embodiments the biological therapy for PG is adalimumab.

The subject may be a human or an animal. In some embodiments the subject is a human. The subject may be from 10 to 50 years, from 15 to 40 years or from 20 to 30 years of age. In some embodiments the subject is, or has previously been, a smoker. In some embodiments the subject has a BMI of at least 30, at least 40 or above.

### Combination Therapies

The compound of the invention, or a formulation or composition comprising the compound, may be used alone to provide a therapeutic effect. The compound of the invention, or a formulation or composition comprising the compound, may also be used in combination with a further therapy.

In some embodiments the further therapy is selected from an anti-androgenic agent, a hormone, an antibiotic (e.g. dapsone, doxycycline, minocycline, clindamycin, rifampin or a carbapenem), a retinoid, an anti-inflammatory agent (including steroids, e.g. corticosteroids, non-steroidal anti-inflammatory agents, sulfasalazine, sodium chromoglycate, 5-aminosalicylic acid, nicotine and colchicine), an analgesic, an immunosuppressive agent (e.g. tacrolimus, azathioprine, mycophenolate, cyclosporin or cyclophosphamide), an antibody (e.g. infliximab), surgery, metformin, a nutritional supplement (e.g. zinc gluconate), a biological therapy (e.g. a TNF-a inhibitor (e.g. adalimumab, infliximab, golimumab or certolizumab pegol), an IL-1 inhibitor (e.g. anakinra), an anti-IL-17 drug (e.g. secukinumab), an anti-IL-23 drug (e.g. risankizumab), or an anti-IL-12/23 drug (e.g. ustekinumab)), a complement C5a inhibitor (e.g. avacopan), a Janus Kinase (JAK) inhibitor (e.g. tofacitinib, INCB054707 or upadacitinib), a leukotriene A4 hydrolase inhibitor (e.g. LYS 006), an IRAK4 degrader (e.g. KT-474), a IRAK4 inhibitor (e.g. PF-06650833), a tyrosine kinase 2 (TYK2) inhibitor (e.g. PF-06826647) or a TYK2/JAK1 inhibitor (e.g. PF-06700841), or any combination thereof.

Such combination treatment may be achieved byway of the simultaneous, sequential or separate dosing of the individual components of the treatment. Such combination products employ the formulation of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of formula (I), or a pharmaceutically acceptable salt, solvate or hydrate thereof present in the formulation of the invention and an approved or otherwise published dosage range(s) of the other pharmaceutically active agent(s).

### Dosages and Dosage Regimens

The dosage and dosing regimen of the formulation of the invention will depend upon a number of factors that may readily be determined by a physician, for example the severity of the disease or condition, the responsiveness to initial treatment, the mode of administration and the particular disease or condition being treated. Examples of suitable doses, dosing volumes and frequencies are set out in the brief summary of the disclosure above.

Suitable modes of administration include oral, intranasal, parenteral (e.g. intravenous, intramuscular, intra-arterial, subcutaneous or intradermal), topical, inhalation (intraorally or intranasally), or a combination thereof. One or more doses may be delivered to the subject via multiple modes of administration.

The total daily dose of the compound of formula (I) administered to the subject may comprise one or more unit doses. The total daily dose may be no more than 120 mg, no more than 100 mg, no more than 80 mg, no more than 60 mg, or no more than 40 mg of the compound of formula (I). In some embodiments, the treatment comprises administering a total daily dose of at least 20 mg, at least 30 mg, at least 40 mg, at least 50 mg, at least 60 mg, at least 80 mg, or at least 100 mg of the compound of formula (I).

In some embodiments the total daily dose administered is from about 5 to about 150 mg, from about 10 to about 120 mg, from about 20 to about 110 mg, from about 30 to about 100 mg, from about 40 to about 90 mg, from about 50 to about 80 mg, or from about 60 to about 70 mg of the compound of formula (I) or salt, solvate or hydrate thereof.

In some embodiments, the treatment comprises administering a unit dose of from about 1 mg to about 100 mg, from about 5 mg to about 70 mg, from about 8 mg to about 65 mg, from about 10 mg to about 60 mg, from about 15 mg to about 50 mg, from about 20 mg to about 45 mg, or from about 30 to about 40 mg. In some embodiments, the treatment comprises administering a unit dose of from about 1 mg to about 50 mg, from about 5 mg to about 40 mg, or from about 10 mg to about 30 mg. In some embodiments, the treatment comprises administering a unit dose of from about 5 mg. In some embodiments, the treatment comprises administering a unit dose of from about 10 mg. In some embodiments, the treatment comprises administering a unit dose of from about 20 mg. In some embodiments, the treatment comprises administering a unit dose of from about 30 mg. In some embodiments, the treatment comprises administering a unit dose of from about 40 mg. In some embodiments, the treatment comprises administering a unit dose of from about 50 mg. In some embodiments, the treatment comprises administering a unit dose of from about 60 mg. In some embodiments, the treatment comprises administering a unit dose of from about 70 mg. In some embodiments, the treatment comprises administering a unit dose of from about 80 mg. In some embodiments, the treatment comprises administering a unit dose of from about 100 mg.

The treatment may be administered from one to four times daily, for example from two to three times daily. In some embodiments the treatment is administered once daily. In some embodiments the treatment is administered twice daily.

Accordingly, in some embodiments the compound of formula (I) is administered in a dose of 5 mg once per day. In some embodiments the compound of formula (I) is administered in a dose of 10 mg once per day. In some embodiments the compound of formula (I) is administered in a dose of 20 mg once per day. In some embodiments the compound of formula (I) is administered in a dose of 30 mg once per day. In some embodiments the compound of formula (I) is administered in a dose of 40 mg once per day. In some embodiments the compound of formula (I) is administered in a dose of 50 mg once per day.

In some embodiments the compound of formula (I) is administered in a dose of 60 mg once per day. In some embodiments the compound of formula (I) is administered in a dose of 70 mg once per day. In some embodiments the compound of formula (I) is administered in a dose of 80 mg once per day. In some embodiments the compound of formula (I) is administered in a dose of 90 mg once per day. In some embodiments the compound of formula (I) is administered in a dose of 100 mg once per day.

In some embodiments the compound of formula (I) is administered in a dose of 5 mg twice per day. In some embodiments the compound of formula (I) is administered in a dose of 10 mg twice per day. In some embodiments the compound of formula (I) is administered in a dose of 20 mg twice per day. In some embodiments the compound of formula (I) is administered in a dose of 30 mg twice per day. In some embodiments the compound of formula (I) is administered in a dose of 40 mg twice per day. In some embodiments the compound of formula (I) is administered in a dose of 50 mg twice per day.

The compound may be administered to the subject over a number of consecutive days or weeks. In some embodiments the compound is administered one or more times daily over a period of at least 10 days, at least 2 weeks, at least 3 weeks, at least 4 weeks, at least 6 weeks, at least 8 weeks, at least 10 weeks, at least 12 weeks, at least 16 weeks, at least 20 weeks, or at least 6 months. In some embodiments, the treatment is administered for no more than 12 months, no more than 10 months, no more than 9 months, no more than 8 months, no more than 6 months, no more than 24 weeks, no more than 20 weeks, no more than 16 weeks, no more than 12 weeks or no more than 10 weeks. For example, the compound may be administered for a period of from 10 days to 20 weeks, from 14 days to 16 weeks, from 21 days to 14 weeks, from 4 to 12 weeks, from 5 to 10 weeks or from 6 to 8 weeks. In some embodiments, the compound is administered to the subject twice daily for up to 4, 6, 10, 16 or 20 weeks. Alternatively, it may be that the treatment is administered for a longer period of time, for example, for maintenance. Thus, in some embodiments the treatment is administered for a period of time of from 6 months to 5 years, from 12 months to 4 years, from 15 months to 3 years, or from 18 to 24 months. In some embodiments treatment is administered for at least 12 weeks, at least 16 weeks, at least 20 weeks, at least 6 months, at least 8 months, at least 10 months or at least 12 months.

It will be appreciated that the dosing period will be determined by the type and severity of the disease being treated. It may be that treatment is continued until the number of inflammatory nodules, abscesses, comedones, ulcers and/or sinus tracts on the subject has been substantially reduced or eliminated.

In one embodiment, the dose of the compound of administered is increased over the first two weeks of treatment. Suitably, the dose is increased from 10 mg twice daily to 30 mg twice daily over the period of two weeks. In some embodiments the compound is administered in an initial total daily dose of about 5 mg to 20 mg and the total daily dose is increased over a period of 1 or 2 weeks. For example the initial daily dose may be 5 mg to 20 mg and this is increased to a daily dose of 50 to 100 mg over a period of 1 or 2 weeks. In some embodiments in an initial total daily dose of about 5 mg to 20 mg and the total daily dose is increased over a period of 1 or 2 weeks to a total daily dose of about 60 to 80 mg per day. In these embodiments the daily dose may be administered as a single daily dose of the compound. However, suitably the total daily dose is administered as a divided dose administered at regular intervals. For example the total daily dose may be administered as substantially equal divided doses 2 to 4 times per day. Suitably the total daily dose is administered as a substantially equal divided dose 2 times per day.

In some embodiments the subject will receive titration of orismilast from 10 mg twice daily to 30 mg twice daily in week 1 followed by 30 mg twice daily ongoing.

In some embodiments the compound is administered for the first two weeks of treatment substantially as described in the “Dose Titration Scheme” in Table 1. During the first 2 weeks there will be a progressive increase in dose until Week 2 (W2) when the subject will be at 30 mg BID. At this point it will be decided if further up-titration to reach 40 mg BID should be initiated. If further up-titration is initiated, the subject will increase the morning dose to 40 mg and maintain the evening dose at 30 mg during the next 2 days. At day 3 after Week 2, the dose will be increased to 40 mg BID and this dose will be maintained until the end of the treatment period.

TABLE 1
Dose titration scheme

| | Day | | | | | | | |
|---|---|---|---|---|---|---|---|---|
| | D 1 | D 1 | D 2 | D 2 | D 3 | D 3 | D 4 | D 4 |
| | AM | PM | AM | PM | AM | PM | AM | PM |
| Dose, mg | 10 | 10 | 10 + 10 | 10 | 10 + 10 | 10 + 10 | 10 + 10 | 10 + 10 |

| | Day | | | | | | | | |
|---|---|---|---|---|---|---|---|---|---|
| | | | | | | | | | From |
| | | | | | | | From | W 2 + 1 | W 2 + |
| | D 5 | D 5 | D 6 | D 6 | D 7 | D 7 | D 8 to | & | until |
| | AM | PM | AM | PM | AM | PM | Week 2 | W 2 + 2 | EoT |
| Dose, mg | 30 | 10 + 10 | 30 | 10 + 10 | 30 | 30 | 30 / 2 | 30 + 10/ | 30 + 10 / |
| | | | | | | | | 30 | 30 + 10 |

EOT = End of Treatment

It will be appreciated that the dose of the formulation and/or the dosage regime may be selected by the skilled person depending on a number of factors such as, but not limited to, the severity of the disease, the age of the subject and/or the presence of any underlying conditions.

### EXAMPLES

The invention is further illustrated by the following examples.

### Example 1: Compositions

Embodiments of compositions for oral administration comprising the compound of formula (I) are shown in Table 2:

TABLE 2
Compositions comprising compound (I) for oral administration

| | | 2: modified | | 4: blend, hard | 5: gastro- |
|---|---|---|---|---|---|
| | 1: hard capsule | release tablet | 3: soft capsule | capsule | resistant capsule |
| core | compound (I) | compound (I) | compound (I) | compound (I) | compound (I) |
| ingredients | | | | | |
| | | Lactose | Triglyceride, | Microcrystalline | Microcrystalline |
| | | monohydrate | medium chain | cellulose | cellulose |
| | | Hypromellose | Lecithin | Lactose | Lactose |
| | | | | monohydrate | monohydrate |
| | | Silica, colloidal | Hard fat | Hypromellose | Coscarmellose |
| | | anhydrous | | | sodium |
| | | Magnesium | Silica, colloidal | Silica, colloidal | Silica, colloidal |
| | | stearate | anhydrous | anhydrous | anhydrous |
| | | | | Magnesium | Magnesium |
| | | | | stearate | stearate |
| shell/coating | Gelatin | Macrogol | Gelatin | Gelatin | Hypromellose |
| | Ferric oxide | PVA | Glycerol | Ferric oxide | Hypromellose |
| | red | | | red | acetate succinate |
| | Titanium | Titanium | Ferric oxide | Titanium | Titanium |
| | dioxide | dioxide | red | dioxide | dioxide |
| | | Talc | Water, purified | | |
| | | Ferric oxide | | | |
| | | yellow | | | |

### Example 2: Inhibition of PDE Enzymes

PDE4 enzymatic activity was measured in a scintillation proximity assay using purified human recombinant PDE4D protein. The results are shown in Table 3:

TABLE 3
Inhibition of PDE4

EC₅₀ values
Compound of formula (I)

| | | |
|---|---|---|
| PDE4D SPA* | 14.7 nM | 7.5 ng/mL |

*SPA = scintillation proximity assay

Inhibition of PDE4 subtypes by the compound of formula (I) was tested using IMAP technology, which is based on the high affinity binding of phosphate by immobilized metal coordination complexes on nanoparticles. The binding reagent complexes with phosphate groups on nucleotide monophosphate generated from cyclic nucleotides (cAMP/cGMP) through phosphodiesterases. With fluorescence polarisation detection, binding causes a change in the rate of the molecular motion of the phosphate bearing molecule, and results in an increase in the fluorescence polarization value observed for the fluorescent label attached to the substrate.

Stocks of the compound of formula (I) were prepared in 100% DMSO. All assays were performed in 3% (final) DMSO for the IMAP assay. The compound was tested in duplicate against the PDE isoform panel at a range of concentrations (10⁻¹⁰, 10⁻⁹, 10⁻⁸, 10⁻⁷ M). The results of the assay are shown in Table 4.

TABLE 4
Inhibition of PDE4 subtypes

| PDE isoform | Concentration of compound (I) (M) | Inhibition (%) |
|---|---|---|
| PDE4A4 | 10⁻⁷ | 47 |
| | 10⁻⁸ | 24 |
| | 10⁻⁹ | 0 |
| | 10⁻¹⁰ | 0 |
| PDE4B2 | 10⁻⁷ | 61 |
| | 10⁻⁸ | 41 |
| | 10⁻⁹ | 18 |
| | 10⁻¹⁰ | 12 |
| PDE4C2 | 10⁻⁷ | 15 |
| | 10⁻⁸ | 2 |
| | 10⁻⁹ | 0 |
| | 10⁻¹⁰ | 9 |
| PDE4D3 | 10⁻⁷ | 74 |
| | 10⁻⁸ | 24 |
| | 10⁻⁹ | 0 |
| | 10⁻¹⁰ | 0 |

The compound of formula (I) inhibited PDE4B and PDE4D most potently, but also PDE4A to a lesser extent. PDE4C was not significantly inhibited at the highest dose tested, 100 nM. The compound of formula (I) inhibited PDE4D splice variant 3 with somewhat higher EC₅₀ value than the inhibition of PDE4D shown in Table 4. This difference is most likely due to different assay techniques used. Without being bound by theory, it is thought that the selectivity of the compound of formula (I) for certain PDE4 subtypes may be beneficial, for example by reducing side effects.

### Inhibition of PDE Enzymes in a Radiometric PDE Assay

The compound of formula (I) (orismilast) and the PDE inhibitors apremilast and roflumilast were assessed in a radiometric PDE assay using a panel of human PDE4 isoforms (PDE4A1, PDE4A4, PDE4A10, PDE4B1, PDE4B2, PDE4B3, PDE4C2, PDE4D1,PDE4D2, PDE4D3, PDE4D4, PDE4D5 and PDE4D7).

Enzyme Preparations: Partially purified human recombinant phosphodiesterases (PDEs) were obtained by cloning cDNA and expressing in S. frugiperda insect cells using a baculovirus expression system. Cells are harvested from culture by centrifuging at 400×g for 5 minutes. Cell pellet is resuspended in 20 ml of RIPA Buffer (150 mM NaCl, 10 mM Tris, 0.1% NP-40, pH8.3), 4 ml/200 ml of culture, plus protease inhibitors (100 μl/10 ml) and incubated on ice for 10-20 minutes then spun at 3500×g for 10 minutes at 4° C. Supernatant is kept and the pellet thrown away.

Radiometric PDE Assay: The assay consists of a two step procedure. Tritium-labelled cyclic AMP is hydrolysed to 5′-AMP o by PDE. The 5′-AMP is then further hydrolysed to adenosine by nucleotidase in snake venom. An anion-exchange resin binds all charged nucleotides and leaves [³H]adenosine as the only labelled compound to be counted by liquid scintillation. The radiometric assay method is a modification of the two-step method described in Thompson et al., Biochemistry 10; 311-316; 1971, adapted for 96 well plate format.

50 μl of diluted PDE enzyme was incubated with 50 μl of [³H]-cAMP (specific activity 25 Ci/mmol) and 11 μl of 50% DMSO (or compound dilution) for 20 minutes at 30° C. The enzyme was diluted in 20 mM Tris HCl pH7.4 (×2.2 final concentration) and [3H]-cAMP was diluted in 10 mM MgCl2, 40 mM Tris HCl pH 7.4 (×2.2 final concentration). Reactions were carried out in Greiner 96 deep well 1 ml master-block. The plates were centrifuged for 9 seconds before the 20 minutes incubation. The reaction was terminated by denaturing the PDE enzyme (at 70° C. for 2 minutes, plates were left to cool on ice for 10 minutes) after which 25 μl snake venom nucleotidase (225 μg/ml final concentration) was added and incubated for 10 minutes (at 30° C.), plates were centrifuged for 9 seconds before incubation. After incubation 200 μl Dowex resin (1×8, 200-400 mesh) was added and the plate shaken for 20 minutes then centrifuged for 3 minutes at 1000×g. 5 μl of supernatant was removed and added to 200 μl of MicroScint-20 in white plates (Greiner 96 well Optiplate) and shaken for 30 minutes before reading on Perkin Elmer TopCount Scintillation Counter. IO₅₀ values were calculated using GraphPad Prism software (version 8).

### Results

The IC₅₀ values (nM) are shown in Table 5:

TABLE 5

| | IC₅₀ (nM) | | |
|---|---|---|---|
| PDE | Apremilast | Orismilast (Compound (I)) | Roflumilast |
| PDE4A1 | 77.98 | 16.39 | 0.45 |
| PDE4A4 | 42.39 | 11.28 | 0.31 |
| PDE4A10 | 341.60 | 52.27 | 1.47 |
| PDE4B1 | 60.56 | 15.68 | 0.56 |
| PDE4B2 | 115.7* | 5.60 | 0.37 |
| PDE4B3 | 116.70 | 2.94 | 0.33 |
| PDE4C2 | 294.7* | 104.20 | 2.31 |
| PDE4D1 | 43.57 | 8.67 | 0.47 |
| PDE4D2 | 53.83 | 2.03* | 0.1539* |
| PDE4D3 | 54.21 | 1.752* | 0.21 |
| PDE4D4 | 41.38 | 2.998* | 0.26 |
| PDE4D5 | 61.36 | 2.177 | 0.24 |
| PDE4D7 | 49.60 | 2.94 | 0.14 |

*value generated by extrapolation of data

### Example 3: Cell-Based TNF-α Release Assay

TNF-α, Serumfree, hPBMC, LPS

Cultures of human PBMC isolated from buffy coats were stimulated with lipopolysaccharide to activate the monocytes and to release TNF-α. PBMC were isolated from fresh buffy coats and frozen for later use. On the assay day, cells were thawed, washed in serum free medium (RPM11640 with 25 mM HEPES, 1% pen/strep, 200 mM L glutamine and 0.5% human serum albumin) and living cells were counted. Test compounds were diluted in DMSO and further diluted in serum-free medium and pipetted into wells of 384 well tissue culture plates. LPS was added to the cells which shortly thereafter were added to the wells with titrated test compounds and incubated for 18 hours at 37° C. The next day, the level of TNF-α in the culture supernatant was quantitated by homogenous time-resolved fluorescence (HTRF) where FRET was measured at 665 nm and normalized to the fluorescence of europium cryptate at 620 nm. The effect (inhibition of TNF-α release) of a test compound was calculated using equation 1:

% Effect=100*(Sx−So)/(Sc−So).

Fluorescence intensities, measured at 665 nm and 620 nm, were used to calculate the 665/620 ratio as an estimate of the level of secreted TNF-α. Sx denotes the 665/620 ratio associated with the test compound. So and Sc denote the 665/620 ratios associated with 0.1% DMSO and 1E-05 M compound (II), respectively.

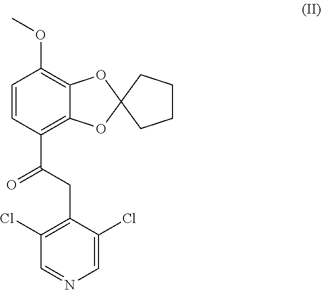 [SMILES: COc1ccc(C(=O)Cc2c(Cl)cncc2Cl)c2c1OC1(CCCC1)O2]

Viability, Serum-Free, hPBMC, LPS

The viability of the cells from the TNF-α assay is measured in the wells using the ATP vialight kit (Perkin Elmer, ATP-lite M, cat. no 6016949). The assay detects the amount of ATP using a bioluminescent method that utilizes an enzyme, luciferase, which catalyses the formation of light from ATP and luciferin according to the following reaction:

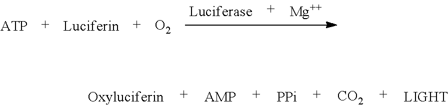

The emitted light intensity is linearly related to the ATP concentration and is measured using a luminometer. The effect (inhibition of viability) of a test compound was calculated using equation 1:

$$\% Effect = 100 * ( Sx - So ) / ( Sc - So )$$

where
So=Emitted light intensity reflecting the ATP concentration in the presence of 0.1% DMSO,
Sc=Emitted light intensity reflecting the ATP concentration in the presence of 10 μM Actinomycin in 0.1% DMSO and
Sx=Emitted light intensity reflecting the ATP concentration in the presence of test compound in 0.1% DMSO.

### Data Analysis

Molar concentrations of an inhibitor that produces 50% of the maximum possible “inhibitory” response (EC50 value), which is associated with the positive ctrl, is calculated according to equation General sigmoidal curve with Hill slope, a to d. This model describes a sigmoidal curve with an adjustable baseline, a. The equation can be used to fit curves where response is either increasing or decreasing with respect to the independent variable, X.

$$\% Effect = \frac{{E}_{max} - {E}_{min}}{1 + {( \frac{C}{Rel {EC}_{50}} )}^{\text{?}}} + {E}_{min}$$

$$\text{?} \text{indicates text missing or illegible when filed}$$

where
C=concentration of test compound
% Effect=normalized response calculated as described in Equation 1
Emin=min response as compound concentration approaches 0
Emax=max response as concentration of tested compound is increasing
EC50=Concentration of tested compound giving a response in the middle of Emin and Emax
nH=Hill coefficient or curve slope.

### Results

The compound of formula (I) was found to be potent inhibitor of monocyte-derived (lipopolysaccharide-induced) TNF-α, as shown in Table 6:

TABLE 6
Effect of compound (I) on TNF-α release
from human peripheral blood mononuclear cells

| | TNF-a assay | | Viability | |
|---|---|---|---|---|
| | EC50, nM | Emax, % | EC50, nM | Emax, % |
| Compound (I) | 11.4 nM | 102 | >9990 | 7% |

### Example 4: Effect of PDE4 Inhibitors on TNF-α Secretion from Human Whole Blood

The effect of the compound of formula (I) and two other PDE4 inhibitors, apremilast and roflumilast N-oxide, were studied in a whole blood TNF-α secretion assay. The JAK inhibitor, Tofacitinib was included as a non-PDE4 inhibitor reference compound. The tested PDE4 inhibitors showed a similar Emax but different EC₅₀ values in the various donors and assays. The compound of formula (I) was shown to be equipotent to—or slightly more potent than—roflumilast-N-oxide and an average of 23 times more potent than apremilast on a molar basis, in both LPS and SEB-induced TNF-α secretion from human whole blood.

### Materials and Methods

In the first experiment, several setups were tested. Freshly drawn human peripheral blood stabilized with heparin was diluted either with X-vivo-15 medium or with RPM11640, and stimulated with different concentrations of lipopolysaccharide (LPS) or Staphylococcus enterotoxin B (SEB) for different time points. The compound of formula (I) was tested at 3 different concentrations in all the different assay setups.

In the second experiment all four compounds were tested in full titration in two donors. Xvivo 15 was used for dilution of the blood and the following four assay versions were run:
LPS 1 μg/mL for 24 hours
LPS 1 μg/mL for 48 hours
SEB 1 μg/mL for 24 hours
SEB 1 μg/mL for 48 hours

Test compounds were diluted in DMSO and added in duplicates to 384 well plates to give a final concentration of 0.1% DMSO in the wells. The blood was diluted with X-vivo 15 medium and added to the wells to give a volume of 80 μL per well and a final dilution of 50% blood/50% medium. The plates were placed in a water bath box in an incubator for 24 or 48 hours. TNF-α in the supernatants was measured by alpha-LISA kits (Perkin Elmer cat. No AL208F).

### Results

Table 7 shows the EC₅₀ values and Emax values obtained with the four compounds in the 4 different assay versions. The Emax was defined as the level of TNF-α in un-stimulated wells.

The Emax for the three PDE4 inhibitors was very similar within one donor and one assay version, but the EC₅₀ value was very different reflecting different potencies of the compounds. The JAK inhibitor, Tofacitinib, induced increased TNF-α secretion in the LPS stimulated assay version but inhibited the SEB stimulated secretion.

TABLE 7
EC₅₀ and Emax values of the test compounds

| | | 24 hours | | | | 48 hours | | | |
|---|---|---|---|---|---|---|---|---|---|
| | | donor 1 | | donor 2 | | donor 1 | | donor 2 | |
| | | EC₅₀ | Emax | EC₅₀ | Emax | EC₅₀ | Emax | EC₅₀ | Emax |
| LPS | Roflumilast | 2.34E−08 | 72 | 2.40E−08 | 79 | 3.86E−08 | 75 | 3.50E−08 | 78 |
| | N-oxide | | | | | | | | |
| | Compound (I) | 5.34E−09 | 76 | 1.84E−08 | 79 | 1.21E−08 | 71 | 1.98E−08 | 80 |
| | Apremilast | 2.10E−07 | 74 | 2.83E−07 | 79 | 2.77E−07 | 77 | 3.64E−07 | 78 |
| | Tofacitinib | Stimulation | | | | Stimulation | | | |
| SEB | Roflumilast | 8.69E−09 | 97 | 3.56E−08 | 66 | 4.77E−09 | 97 | 3.34E−08 | 80 |
| | N-oxide | | | | | | | | |
| | Compound (I) | 4.93E−09 | 97 | 2.49E−08 | 66 | 4.66E−09 | 98 | 4.78E−08 | 83 |
| | Apremilast | 1.39E−07 | 98 | 6.20E−07 | 65 | 1.55E−07 | 97 | 5.73E−07 | 80 |
| | Tofacitinib | 1.68E−07 | 98 | 8.30E−06 | 100* | 1.82E−07 | 95 | 1.45E−06 | 102 |

*Locked value, no top plateau

Table 8 shows the mean EC₅₀ value upon pooling the results from all eight whole blood TNF-α tests.

TABLE 8
Geometric mean of EC₅₀ values across different assay parameters

| | Mol/L | ng/mL |
|---|---|---|
| Apremilast EC₅₀ mean of 8 determinations | 2.87 E−07 | 132 |
| Compound (I) EC₅₀ mean of 8 determinations | 1.26 E−08 | 6.43 |
| Roflumilast N-oxide EC₅₀ mean of 8 determinations | 2.10 E−08 | 8.80 |

Based on the mean EC₅₀ values shown in Table 8, the compound of formula (1) is 23 times more potent than apremilast based on molar concentrations and 21 times more potent based on ng/mL concentrations. The compound of formula (1) is 1.7 times (M)/1 0.4 times (ng/mL) more potent than Roflumilast.

### Example 5: Human Whole Blood Cytokine Secretion Assay

TruCulture blood collection and incubation tubes were tested with fresh human whole blood with or without addition of 20 nM of the compound of formula (1). The best results were obtained using heparin-stabilised blood and anti-CD3/CD28 test tubes. The secretion of IFN-γ, IL-1β, IL.-2, IL-4 and TNF-α was inhibited by the compound of formula (I) (Table 9), but IL-8 and IL-13 were not clearly inhibited.

TABLE 9
Inhibition of cytokines from whole blood

| % inhibition at 20 nM ^{a} | IFN-γ | IL-1 | IL-2 | IL-4 | TNF-α |
|---|---|---|---|---|---|
| Compound (I) | 54% | 40% | 50% | 44% | 60% |

^{a} Mean of 3 experiments.

The cytokine inhibition profile of the compound of formula (I) suggests that it will be beneficial for the treatment of inflammatory skin diseases. The compound potently inhibits the secretion of TNF-α and IL-1β, two cytokines that are highly associated with inflammation. The compound also inhibits IFN-γ, IL-2 and IL-4. IFN-γ is a T-cell derived Th1 cytokine that plays a role in Th1 immune responses.

### Example 6: PDE4 Inhibitors in the Chronic Oxazolone Model in BALB/cA Mice

The purpose of the study was to evaluate three different oral doses of the compound of formula (I) and the reference compound apremilast at one dose in the chronic oxazolone model.

### Materials and Methods

### Dosing Preparations

Four vials were delivered per treatment group (group 3-5). Every 3rd day, vehicle (1% methylcellulose) was added to the appropriate vials before dosing (see Table 10 below). The solution was stable for at least 7 days, but was made fresh every 3 days. Vials were delivered for treatment group 6. Every day vehicle (1% methylcellulose) was added just before dosing. It was ensured that the compound was dissolved completely before dosing. Dosing volume for all groups was 10 ml/kg or 0.01 ml/g bw.

TABLE 10
Dosing

| | | Dose | Vial | Vehicle | Final | |
|---|---|---|---|---|---|---|
| Group | Compound | (mg/kg bw) | (mg) | (ml) | concentration | |
| 3 | (I) | 10 | 9.6 | 9.6 | 1 | mg/ml |
| 4 | (I) | 3 | 3.0 | 10.0 | 0.3 | mg/ml |
| 5 | (I) | 1 | 0.9 | 9.0 | 0.1 | mg/ml |
| 6 | Apremilast | 30 | 10 | 3.33 | 3 | mg/ml |

Dexadresone was prepared each day by suspending 0.3 ml dexadresone vet (2 mg/ml) in 2.7 ml vehicle (1% methylcellulose) immediately before dosing. Final concentration was 0.2 mg/ml. The compound was fully suspended before dosing.

Animals used were female BALB/cABomTac mice, 7 weeks of age.

Mice in groups 2-7 were sensitized with oxazolone in acetone (0.8%) by applying 10 μl of the solution to each side of the right ear on day-7. Additionally, on day-7 mice in group 1 were dosed with 10 μl of acetone on each side of the left AND right ear. The 0.8% oxazolone solution is prepared as shown in Table 11 below:

TABLE 11
0.8% Oxazolone solutions

| No. of animals | Oxazolone (mg) | Acetone (ml) | Concentration |
|---|---|---|---|
| 50 | 12 | 1.5 | 0.80% |
| 60 | 14.4 | 1.8 | 0.80% |
| 70 | 16.8 | 2.1 | 0.80% |
| 80 | 19.2 | 2.4 | 0.80% |
| 90 | 21.6 | 2.7 | 0.80% |
| 100 | 24 | 3 | 0.80% |

One week after sensitization the mice in groups 2-7 were challenged for the first time with 0.4% oxazolone in acetone. Specifically, mice were dosed with 10 μl on each side of the right ear. Dosing was done at the following days: day 0, 3, 5, 7, 10, 12, 14, 16, 18 and 20.

On the exact same days, mice in group 1 were dosed with 10 μl acetone on each side of the left AND right ear. The 0.4% oxazolone solution was prepared as shown in Table 12 below:

TABLE 12
0.4% Oxazolone solutions

| No. of animals | Oxazolone (mg) | Acetone (ml) | Concentration |
|---|---|---|---|
| 50 | 6 | 1.5 | 0.40% |
| 60 | 7.2 | 1.8 | 0.40% |
| 70 | 8.4 | 2.1 | 0.40% |
| 80 | 9.6 | 2.4 | 0.40% |
| 90 | 10.8 | 2.7 | 0.40% |
| 100 | 12 | 3 | 0.40% |

Oxazolone: 4-Ethoxymethylene-2-phenyl-2-oxazolin-5-one (≥90% (HPLC)), Sigma Aldrich, catalogue number E0753-5G, batch number 039K3533.

Mice were randomized according to the ear-thickness measured on day 10. The purpose was to create groups with similar mean ear thickness and standard deviations. The induced phenotype was treated orally with test compound as shown in Table 13 below. In group 2-6, 10 ml/kg bw of compound dissolved in methylcellulose (or methylcellulose only for group 2) was dosed orally once daily. Animals in group 1 were dosed orally with 10 ml/kg methylcellulose. All mice are treated from day 10 to day 21—both days included.

TABLE 13
Treatment schedule

| | No. of | | Concentration | Times | Volume p.o. | Dose |
|---|---|---|---|---|---|---|
| Group | animals | Compound | (mg/ml) | Daily | (ml/g bw) | (mg/kg) |
| 1 | 4 | Methylcellulose | NA | 1 | 0.01 | NA |
| 2 | 10 | Methylcellulose | NA | 1 | 0.01 | NA |
| 3 | 10 | Compound (I) | 1 | 1 | 0.01 | 10 |
| 4 | 10 | Compound (I) | 0.3 | 1 | 0.01 | 3 |
| 5 | 10 | Compound (I) | 0.1 | 1 | 0.01 | 1 |
| 6 | 10 | Apremilast | 3 | 1 | 0.01 | 30 |
| 7 | 10 | Dexadresone | 0.2 | 1 | 0.01 | 2 |

### Sampling

Blood and the right ear were sampled from all animals. Blood was sampled by cardiac puncture from animals anaesthetized with isoflurane (3% in oxygen). When the mice were fully anaesthetized (no interdigital reflex present), blood was sampled with a 25 G needle and a 1 ml syringe and transferred to 2.5 ml BD SST Vacutainer® tubes. After sampling vials were placed on ice for 30 minutes before being centrifuged for 10 min at 1000 G at 4° C. Immediately afterwards, serum was transferred to 1.4 ml noncoded u-bottom bulk Micronics tubes and stored at −80° C. Mice were euthanized by cervical dislocation immediately after blood sampling, while they were still fully anaesthetized.

Tissue from side A of the ear was placed in a Nunc cryotube and snap-frozen in liquid nitrogen. Samples were stored at −80° C. and/or sent to DMPK for analysis of drug concentration. Samples were taken 2 hours after the last application of treatment compound.

Tissue from side B of the ear was placed in a Nunc round-bottom cryotube and snap-frozen in liquid nitrogen. Samples were stored at −80° C. and/or sent to Molecular Biomedicine (Lili Rohde/Paola Lovato) for cytokine analysis. Samples were taken 2 hours after the last application of test compound.

Ear thickness was obtained online (directly into excel-spreadsheet) on day 10, 12, 14, 17, 19 and 21 with an Absolute Digimatic micrometer (Mitutoyo, Aurora, IL, ID-C1012CB code 543-274B). Right and left ears were measured on animals in group 1 and only the right ear was measured on animals from group 2 to 7. Ear measurements were performed before dosing, except on the day of termination where the ears were measured 2 hours after the last dosing. The relative ear-thickness was calculated by subtracting the mean ear-thickness of group 1 from the measured ear-thickness of animals in groups 2 to 7.

The compound concentration in serum was measured from animals in groups 2 to 6.

Statistical methods: One-way ANOVA with Dunnetts' post-test (compared to vehicle group=group 2); significance level 0.05.

### Results

As shown in FIG. 2, the ear thickness AUC of all dosed groups was significantly lower than AUC of the vehicle group. Blood was drawn from the animals 1 hour after the last dose at the expected Cmax. As shown in FIG. 3, the compound of formula (I) reached a similar reduction of ear thickness using a much lower dose (30-fold) and achieving much lower serum concentrations (>20 fold) compared to apremilast. Without being bound by theory, this could indicate that compound (I) may reach a similar efficacious outcome having 20-times lower systemic exposure and thus using a lower dose.

### Example 7: Emesis Study in Ferrets

The objectives of this study were to assess the emetogenic properties of the compound of formula (I) and roflumilast in the conscious, unrestrained ferret. Efficacy in the ferrets was measured as well, by analysing LPS-induced TNF-α ex vivo in whole blood after single oral administration. E. coli LPS (3 mg/kg) was injected ip 1.5 hours after oral administration of compounds, and TNF-α was measured 1 and 3 hours after LPS injection. TNF-α levels were determined in serum using a commercial ELISA kit.

The compounds were administered orally at doses of 1, 3, 10 and 30 mg/kg, using 1% (w/v) methylcellulose 400 centipoises in water as vehicle. Fasted animals were dosed and observed, with a minimum wash-out of 4 days between two sessions. After treatment, animals were singly housed and emetic episodes and associated prodromic signs (licking, mouth scratching, yawning, “wet dog” shakes, backward walking) were counted for approximately 3 hours. Following the emesis experiment blood samplings were performed at selected time points for serum clinical chemistry and pharmacodynamics.

### Results

The compound of formula (I) was found not to influence the health status or body weight gain of the ferrets. Emetogenic properties were absent at dose levels of 1 and 3 mg/kg and were observed at 10 and 30 mg/kg, in a dose-related manner. Therefore, the ‘no observable adverse effect level’ (NOAEL) for the compound of formula (I) in the conscious, unrestrained ferret is 3 mg/kg.

The compound of formula (I), administered at dose levels of 1, 3 and 10 mg/kg, attenuated, in a clear dose-related manner, the increase of TNF-α levels observed at 1 hour. A return towards baseline values was observed 3 hours after LPS administration.

Roflumilast, administered at dose levels of 1, 3, 10 and 30 mg/kg, did not influence the health status or body weight gain of the ferrets. Emetogenic properties were absent at the dose level of 1 mg/kg and were dose dependently observed at dose levels from 3 to 30 mg/kg. Therefore, the ‘no observable adverse effect level’ (NOAEL) for roflumilast in the conscious, unrestrained ferret is 1 mg/kg.

Roflumilast, administered at dose levels of 1 and 3 mg/kg, attenuated in a dose-related manner, the increase of TNF-α levels observed at 1 hour. A return towards baseline values was observed 3 hours after LPS administration.

Overall, the conclusion from this study is that the compound of formula (I) has a more favourable profile than Roflumilast, as the safety margin in ferrets is 3-fold higher than that of Roflumilast.

### Example 8: Clinical Study

The efficacy of the peroral anti-inflammatory PDE4-inhibitor Orismilast was investigated in two patients, one with pyoderma gangrenosum and concomitant HS, the other with PASH syndrome. Patients received a titrated dose of Orismilast of up to 40 mg twice daily for 16 weeks.

### Results

Case 1: A 50-year-old obese man known with severe affection of Pyoderma gangrenosum, Acne and HS (PASH syndrome) was treated with Orismilast 20 mg twice daily. His HS lesions gradually improved and pyoderma wounds slowly recovered with new granulation tissue and increasing epithelization.

Case 2: A 49-year-old normal-weight woman with large pyoderma wounds in both groins after surgical removal of HS tunnels as well as active HS in other areas was gradually titrated to Orismilast 30 plus 40 mg daily. During treatment, her HS lesions remained calm and the pyoderma wounds completely healed. One month after discontinuation, the patient experienced a flare-up and anti-inflammatory treatment had to be re-introduced.

The results indicate that the PDE4-inhibitor Orismilast ameliorates recalcitrant pyoderma gangrenosum lesions in patients with concomitant HS.

1-29. (canceled)

30. A method of treating neutrophilic dermatoses in a subject, the method comprising administering to the subject a therapeutically effective amount of a compound of formula (I):

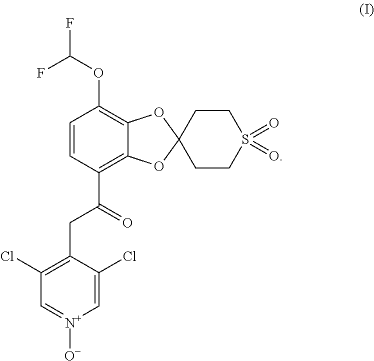 [SMILES: O=C(Cc1c(Cl)c[n+]([O-])cc1Cl)c1ccc(OC(F)F)c2c1OC1(CCS(=O)(=O)CC1)O2]

31. The method of claim 30, wherein the method reduces pain caused by or associated with neutrophilic dermatoses.

32. The method of claim 30, wherein the method reduces inflammation caused by or associated with neutrophilic dermatoses.

33. The method of claim 30, wherein the method eliminates, promotes healing of, or reduces the severity, spread, size, depth, and/or growth rate of ulcers or sores caused by or associated with neutrophilic dermatoses, and/or prevents the development of additional ulcers or sores associated with neutrophilic dermatoses.

34. The method of claim 30, wherein the neutrophilic dermatoses is not solely hidradenitis suppurativa (HS).

35. The method of claim 30, wherein the neutrophilic dermatoses is selected from pyoderma gangrenosum, Sweet's syndrome, Sneddon-Wilkinson disease (also known as subcorneal pustular dermatosis), Behget disease, neutrophilic panniculitis, neutrophilic eccrine hidradenitis, erythema elevatum et diutinum, neutrophilic urticaria, Group of IgA neutrophilic dermatoses, amicrobial pustulosis of the folds, Hallopeau's continuous acrodermatitis, acute generalised exanthematus pustulosis, infantile acropustulosis, aseptic abscesses, PASH syndrome (pyoderma gangrenosum, acne and hidradenitis suppurativa), PAPA syndrome (pyoderma gangrenosum, acne and pyogenic arthritis), PASS syndrome (pyoderma gangrenosum, acne conglobate, hidradenitis suppurativa, seropositive spondyloarthropathies), PAPASH syndrome (pyoderma gangrenosum, pyogenic arthritis, acne, hidradenitis suppurativa), PsAPASH syndrome (psoriatic arthritis, pyoderma gangrenosum, acne, hidradenitis suppurativa) histiocytoid neutrophilic dermatitis, neutrophilic dermatitis of the dorsal hands, bowel bypass syndrome (bowel-associated dermatitis-arthritis syndrome), palisading neutrophilic granulomatous dermatitis, VEXAS syndrome, or any combination thereof.

36. The method of claim 30, wherein the neutrophilic dermatoses is pyoderma gangrenosum.

37. The method of claim 36, wherein the pyoderma gangrenosum is selected from classic pyoderma gangrenosum, peristomal pyoderma gangrenosum, atypical/bullous pyoderma gangrenosum, pustular pyoderma gangrenosum, vegetative pyoderma gangrenosum, pathergic pyoderma gangrenosum, necrotizing-fasciitis-like pyoderma gangrenosum, malignant pyoderma gangrenosum, peristomal pyoderma gangrenosum, and post-operative pyoderma gangrenosum.

38. The method of claim 36, wherein the subject has concomitant hidradenitis suppurativa.

39. The method of claim 30, wherein the neutrophilic dermatoses is PASH syndrome.

40. The method of claim 30, wherein the subject does not have HS.

41. The method of claim 30, wherein said treatment comprises oral, topical, and/or intravenous administration of the compound of formula (I).

42. The method of claim 30, wherein the compound is administered to the subject for at least 4 weeks.

43. The method of claim 30, wherein the treatment comprises administering the compound of formula (I) in a total daily dose of no more than 80 mg.

44. The method of claim 30, wherein the compound is administered twice daily.

45. The method of claim 30, wherein the treatment comprises:
(i) administering the compound in a dose of 20 mg twice per day;
(ii) administering the compound in a dose of 30 mg twice per day; or
(iii) administering the compound in a dose of 40 mg twice per day.

46. The method of claim 30, wherein the compound is administered orally.

47. The method of claim 30, wherein the compound is comprised within a modified-release formulation.

48. The method of claim 47, wherein the modified-release formulation releases a mean amount of from about 11% to about 65% of the compound of formula (I) after 45 minutes and more than 80% of the compound of formula (I) after 180 minutes when placed in a dissolution medium of 900 ml 0.5% sodium dodecyl sulfate in 0.1N HCl using Ph. Eur. 2.9.3 Apparatus II and a paddle speed of 75 rpm.

49. The method of claim 47, wherein the modified-release formulation comprises the compound of formula (I) and a hydrophilic matrix former.

50. The method of claim 49, wherein the hydrophilic matrix former comprises hydroxypropyl methyl cellulose.

51. The method of claim 47, wherein the modified-release formulation comprises the compound of formula (I) and 15% w/w to 25% w/w of hydroxypropyl methyl cellulose.

52. The method of claim 30, wherein the compound is formulated for topical administration.

53. The method of claim 30, wherein the subject is a human.

54. The method of claim 30, wherein the subject is suffering from a comorbidity selected from obesity, metabolic syndrome, inflammatory bowel disease, spondyloarthropathy, inflammatory arthritis, a haematological malignancy, hepatitis, blood dyscrasia, glanulomatosis with polyangiitis, psoriasis, atopic dermatitis, or any combination thereof.

55. The method of claim 30, wherein the compound of formula (I) is administered in combination with a further therapy.

56. The method of claim 55, wherein the further therapy is selected from: an anti-androgenic agent, a hormone, an antibiotic, a retinoid, an anti-inflammatory agent, an analgesic, an immunosuppressive agent, an antibody, surgery, metformin, a nutritional supplement, a biological therapy for HS, a complement C5a inhibitor, a Janus Kinase (JAK) inhibitor, a leukotriene A4 hydrolase inhibitor, an IRAK4 degrader, a IRAK4 inhibitor, a tyrosine kinase 2 (TYK2) inhibitor, a TYK2/JAK1 inhibitor, or any combination thereof.

57. The method of claim 55, wherein the further therapy is selected from a TNF-a inhibitor, an IL-1 inhibitor, an anti-IL-17 drug, an anti-IL-23 drug, an anti-IL-12/23 drug, or any combination thereof.

58. The method of claim 30, wherein said treatment provides selective inhibition of PDE4D and/or PDE4B.

59. The method of claim 30, wherein said treatment provides selective inhibition of PDE4D3 and/or PDE4B2.
